SEQUENCE LISTING (30 sequences)

SEQ ID NO: 1 - protein, 16 aa, synthetic construct
  VARIANT 8: note = May be acetylated
FINSENFKVCDEPHPL

SEQ ID NO: 2 - protein, 17 aa, synthetic construct
  VARIANT 9: note = May be acetylated
NANIDEAYKILAHLWHL

SEQ ID NO: 3 - protein, 17 aa, synthetic construct
  VARIANT 9: note = May be acetylated
TFQMAEYLKLEFIKEIG

SEQ ID NO: 4 - protein, 16 aa, synthetic construct
  VARIANT 9: note = May be acetylated
GLLARLCQKTMAPVAS

SEQ ID NO: 5 - protein, 17 aa, synthetic construct
  VARIANT 9: note = May be acetylated
IAGPPGTGKTTSILCLA

SEQ ID NO: 6 - protein, 17 aa, synthetic construct
  VARIANT 9: note = May be acetylated
IDVVRNKIKMFAQQKVT

SEQ ID NO: 7 - protein, 17 aa, synthetic construct
  VARIANT 9: note = May be acetylated
KIKMFAQQKVTLPKGRH

SEQ ID NO: 8 - protein, 17 aa, synthetic construct
  VARIANT 9: note = May be acetylated
NANIDEAYKILAHLWHL

SEQ ID NO: 9 - protein, 17 aa, synthetic construct
  VARIANT 9: note = May be acetylated
IGNIFRVCKTFQMAEYL

SEQ ID NO: 10 - protein, 17 aa, synthetic construct
  VARIANT 9: note = May be acetylated
EYLKLEFIKEIGYTHMK

SEQ ID NO: 11 - protein, 17 aa, synthetic construct
  VARIANT 9: note = May be acetylated
RALLGPALKDAMLELNA

SEQ ID NO: 12 - protein, 15 aa, synthetic construct
DPAPAFSKAPGSAGH

SEQ ID NO: 13 - protein, 15 aa, synthetic construct
AGPPGTGKTTSILCL

SEQ ID NO: 14 - protein, 15 aa, synthetic construct
ALLGPALKDAMLELN

SEQ ID NO: 15 - protein, 15 aa, synthetic construct
IKMFAQQKVTLPKGR

SEQ ID NO: 16 - protein, 15 aa, synthetic construct
TLPKGRHKIIILDEA

SEQ ID NO: 17 - protein, 15 aa, synthetic construct
RTMEIYSKTTRFALA

SEQ ID NO: 18 - protein, 15 aa, synthetic construct
CAVLRYTKLTDAQIL

SEQ ID NO: 19 - protein, 15 aa, synthetic construct
LLARLCQKTMAPVAS

SEQ ID NO: 20 - protein, 17 aa, synthetic construct
IAGPPGTGKTTSILCLA

SEQ ID NO: 21 - protein, 17 aa, synthetic construct
TFQMAEYLKLEFIKEIG

SEQ ID NO: 22 - protein, 17 aa, synthetic construct
WVEKYRPVKLNEIVGNE

SEQ ID NO: 23 - protein, 17 aa, synthetic construct
TRLMNVIEKERVPYTDD

SEQ ID NO: 24 - protein, 17 aa, synthetic construct
NANIDEAYKILAHLWHL

SEQ ID NO: 25 - protein, 17 aa, synthetic construct
RALLGPALKDAMLELNA

SEQ ID NO: 26 - protein, 17 aa, synthetic construct
HYELPWVEKYRPVKLNE

SEQ ID NO: 27 - protein, 17 aa, synthetic construct
RRTMEIYSKTTRFALAC

SEQ ID NO: 28 - protein, 17 aa, synthetic construct
FINSENVFKVCDEPHPL

SEQ ID NO: 29 - protein, 17 aa, synthetic construct
EYLKLEFIKEIGYTHMK

SEQ ID NO: 30 - protein, 17 aa, synthetic construct
RCAVLRYTKLTDAQILT

(19) United States
(12) Patent Application Publication
(10) Pub. No.: US 2025/0162990 A1
(43) Pub. Date: May 22, 2025

(54) COMPOUNDS AND METHODS FOR INHIBITING CANCERS OVER-EXPRESSING REPLICATION FACTOR C 40

(71) Applicant: RAADYSAN BIOTECH, INC., Fishkill, NY (US)

(72) Inventors: Rakhee GUPTE, Fishkill, NY (US); Steven TAYLOR, Fishkill, NY (US)

(21) Appl. No.: 18/834,974

(22) PCT Filed: Jan. 23, 2023
(86) PCT No.: PCT/US2023/011336

§ 371 Date: Jul. 31, 2024

Related U.S. Application Data
(60) Provisional application No. 63/305,381, filed on Feb. 1, 2022.

(51) Int. Cl. C07D 209/08; A61K 31/381; A61K 31/4045; A61P 35/00; C07D 333/38

(52) U.S. Cl. CPC C07D 209/08; A61K 31/381; A61K 31/4045; A61P 35/00; C07D 333/38

(57) ABSTRACT

Compounds and methods of inhibiting the expression and/or activity of Replication Factor C40 in cancer cells and of treating cancers expressing Replication Factor C 40 are described herein

### CROSS-REFERENCE TO RELATED APPLICATIONS

This application is a U.S. National Phase Application under 35 U.S.C. § 371 of International Patent Application No. PCT/US2023/011336, filed Jan. 23, 2023, and claims priority to U.S. Provisional Patent Application No. 63/305,381, filed Feb. 1, 2022, which are incorporated herein by reference in their entireties.

### SEQUENCE LISTING

The instant application contains a Sequence Listing which has been submitted electronically in XML format and is hereby incorporated by reference in its entirety. Said XML copy, created on Aug. 16, 2024 is named 10501_010398-US1_SL2.xml and is 30.6 bytes in size.

### FIELD OF THE INVENTION

The present invention relates to compounds and methods of inhibiting the expression and/or activity of Replication Factor C 40 in cancer cells and of treating or alleviating cancers expressing Replication Factor C 40.

### BACKGROUND

DNA replication requires the collaborative involvement of a formidable number of proteins.¹⁰ In eukaryotes, several accessory proteins such as Replication Factor C (RFC) and Proliferating Cell Nuclear Antigen (PCNA), confer speed and high processivity to the replicative polymerases (DNA polymerases δ and ε). The RFC complex consists of five subunits—RFC140, RFC40, RFC38, RFC37 and RFC36¹ and its assembly commits the cell to DNA replication by loading PCNA onto DNA. It is involved in many DNA transactions such as DNA damage checkpoint response, maintenance of genomic stability and regulation of sister chromatid cohesion in mitosis as well as in meiosis.²⁻³

Importantly, amongst all the RFC subunits, RFC40, which is also known as RFC2, is the only subunit that possesses an ATPase activity that is critical for the functioning of the RFC complex, such as unloading PCNA and inhibiting DNA Pol δ activity by itself.⁴⁻⁵ Additionally, it has been previously reported that RFC40 is required for accurate chromosomal segregation and cytokinesis. This core DNA replication protein is evolutionarily conserved, in various tissues as well as cross different species, and is important for cell survival.⁶

Although cancer is a result of de-regulated DNA replication and cell proliferation, the role of aberrant expression of proteins directly involved in DNA replication has not been well characterized. RFC40 has been reported to be over-expressed in choriocarcinoma,⁷ acute and chronic myeloid leukemia,⁸⁻⁹ nasopharyngeal cancer¹⁰ and glioblastomas¹¹⁻¹². It has been demonstrated that higher expression of RFC40 has been associated with lower overall survival and disease-free survival in patients with hepatocellular carcinoma¹³. RFC40 protein and/or gene over-expression has been disclosed in U.S. Pat. No. 9,970,012 to be a non-receptor based molecular marker and specific target for breast cancer, irrespective of its receptor status, including estrogen sensitive, Human epidermal growth factor receptor 2(HER2/neu/ERBB2) positive and triple negative breast cancer (TNBC). RFC40 protein and messenger RNA encoding it, as well as RFC40 gene copy numbers, are increased in breast cancers, including in estrogen sensitive, HER2 positive and TNBC.

Breast cancer accounts for 18% of all cancers in women, making it the second highest cause of cancer-related deaths in women.¹⁴ Early diagnosis and treatment of breast cancer plays an enormous role in reducing deaths.¹⁵ The effect of over-expression of endocrine (estrogen, ER; progesterone, PR) and growth factor (HER2) receptors in breast cancer have been extensively studied.³ Therefore, most of the drugs available for the treatment of breast cancers, target either the endocrine (ER) or growth factor (ErbB-1, ErbB-2/HER2, ErbB-3, and ErbB-4) receptors for therapy.¹⁶ However, triple negative breast cancer (TNBC) does not respond to these therapies since it lacks all these receptors. In addition, complex heterogeneity (currently 6 subtypes of TNBC have been identified)¹⁷ and ethnic disparity¹⁸⁻¹⁹, associated with TNBC, further complicates its treatment options, restricting it to radiation, chemotherapy, and surgery. Although newer drugs such as combinations of chemotherapy+PD-L1 (immunotherapy)²⁰ and/or PARP (DNA repair)²¹⁻²² inhibitors have been tried for the treatment of TNBC, they do not increase the survival rates beyond 2-5 months, leaving an appalling deficit for the availability of effective therapies for the treatment of TNBC.

With limited access to treatment for patients with TNBC, many oncologists have voiced their limitations and frustration for lack of tools to treat this deadly disease and hope that new drugs and newer approaches, will equip them with options that will offer hope to the TNBC patients and improve survival rates.²³⁻²⁵ Additionally, its aggressive nature, poor prognosis, relative low survival rates, and sizable patient population, heterogenicity and ethnic disparity, makes it imperative to expedite the development of targeted therapies for these patients.

There is an ongoing need for better treatments of cancers that inhibit cancer cell proliferation without affecting normal cells in mammals afflicted with these cancers, and in particular, for treating cancers expressing RFC40.

### SUMMARY OF THE INVENTION

The present invention relates to compounds of Formulas I to VIII, pharmaceutical compositions comprising the compounds, methods of using the compounds to inhibit the expression and/or activity of RFC40 in cancer cells in a subject, and methods of using the compounds to ameliorate or treat RFC40-mediated cancers in a subject.

The present invention is directed to a compound of Formula I

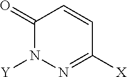

including all pharmaceutically acceptable salts, crystalline forms, stereoisomers, prodrugs, and amorphous forms; wherein X is selected from the group consisting of aryl and heteroaryl; wherein each X is optionally substituted with one or more substituents selected from the group consisting of H, halogen, alkyl, hydroxy, alkoxy, alkenyloxy, alkynyloxy, amido, alkylthiol, alkylsulfoxide, alkylsulfone, alkylthioester, and heteroaryl; wherein two adjacent substituents on X may be taken together to form a cycloalkyl, cycloalkenyl, heterocyclyl, and heteroaryl; and Y is alkyl, wherein Y is optionally substituted with one or more substituents selected from the group consisting of alkyl, alkenyl, alkynyl, amido, carbonyl, carboxyl, aryl, and heteroaryl; wherein two adjacent substituents on Y may be taken together to form a cycloalkyl, cycloalkenyl, heterocyclyl, and heteroaryl.

In an embodiment of the invention, preferred groups of compounds of Formula I include those compounds wherein Y is substituted with one or more substituents selected from the group consisting of amido and carboxyl. The amido may be substituted with one or more substituents selected from the group consisting of alkyl carboxylic acid, aryl, and heteroaryl. The carboxyl may be a carboxylic acid group.

The present invention is directed to a compound of Formula II

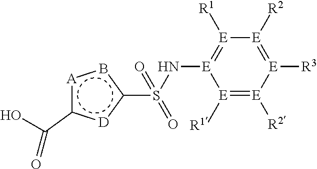

including all pharmaceutically acceptable salts, crystalline forms, stereoisomers, prodrugs, and amorphous forms, wherein A is selected from the group consisting of CR′, CR′═CH, CR′═N, NH, N, S, and O; B is selected from the group consisting of CR′, CR′═CH, NH, N, S, and O; D is selected from the group consisting of CR′, NH, N, S, and O; E at each instance is independently selected from the group consisting of C and N; R′ at each instance is independently selected from the group consisting of H, alkyl, alkenyl, alkynyl, hydroxy, alkoxy, and halogen; R¹ and R¹′ are each individually selected from the group consisting of H, alkyl, alkenyl, alkynyl, hydroxy, alkoxy, alkenyloxy, alkynyloxy, alkylthiol, alkylsulfoxide, alkylsulfone, alkylthioester, halogen, carboxylate, amido, aryl, and heteroaryl; R² and R²′ are each individually selected from the group consisting of H, alkyl, alkenyl, alkynyl, hydroxy, alkoxy, alkenyloxy, alkynyloxy, alkylthiol, alkylsulfoxide, alkylsulfone, alkylthioester, halogen, carboxylate, amido, aryl, and heteroaryl; and R³ is selected from the group consisting of H, alkyl, alkenyl, alkynyl, alkoxy, hydroxy, alkenyloxy, alkynyloxy, alkylthiol, alkylsulfoxide, alkylsulfone, alkylthioester, halogen, carboxylate, amido, aryl, and heteroaryl; wherein two of R¹, R¹′, R², R²′, and R³ may be taken together to form a cycloalkyl, heterocyclo, aryl, and heteroaryl.

In an embodiment of the invention, preferred groups of compounds of Formula II include those compounds wherein (1) A is CR′, wherein R′ is H; B is S; and D is CR′, wherein R′ is H; (2) A is NH; B is CR′, wherein R′ is H; and D is CR′, wherein R′ is H; (3) A is CR′═CH, wherein R′ is H; B is CR′, wherein R′ is H; and D is CR′, wherein R′ is H; and (4) A is CR′, wherein R′ is H; B is CR′, wherein R′ is H; and D is O.

The present invention is directed to a compound of Formula III

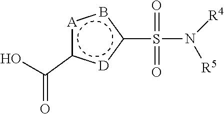

including all pharmaceutically acceptable salts, crystalline forms, stereoisomers, prodrugs, and amorphous forms, wherein A is selected from the group consisting of CR′, CR′═CH, NH, N, S, and O; B is selected from the group consisting of CR′, CR′═CH, NH, N, S, and O; D is selected from the group consisting of CR′, NH, N, S, and O; and R⁴ and R⁵ may be taken together to form a 5, 6, or 7 membered optionally substituted heterocyclo or a 5, 6, or 7 membered optionally substituted heteroaryl.

The present invention is directed to a compound of Formula IV

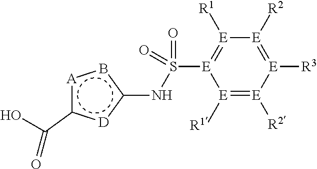

including all pharmaceutically acceptable salts, crystalline forms, stereoisomers, prodrugs, and amorphous forms, wherein A is selected from the group consisting of CR′, CR′═CH, CR′═N, NH, N, S, and O; B is selected from the group consisting of CR′, CR′═CH, CR′═N, NH, N, S, and O; D is selected from the group consisting of CR′, NH, N, S, and O; E at each instance is independently selected from the group consisting of CR′, NH and N; R′ at each instance is independently selected from the group consisting of H, alkyl, alkenyl, alkynyl, hydroxy, alkoxy, and halogen; R¹ and R¹′ are each individually selected from the group consisting of H, alkyl, alkenyl, alkynyl, hydroxy, alkoxy, alkenyloxy, alkynyloxy, alkylthiol, alkylsulfoxide, alkylsulfone, alkylthioester, halogen, carboxylate, amido, aryl, and heteroaryl; R² and R²′ are each individually selected from the group consisting of H, alkyl, alkenyl, alkynyl, hydroxy, alkoxy, alkenyloxy, alkynyloxy, alkylthiol, alkylsulfoxide, alkylsulfone, alkylthioester, halogen, carboxylate, amido, aryl, and heteroaryl; and R³ is selected from the group consisting of H, alkyl, alkenyl, alkynyl, alkoxy, hydroxy, alkenyloxy, alkynyloxy, alkylthiol, alkylsulfoxide, alkylsulfone, alkylthioester, halogen, carboxylate, amido, aryl, and heteroaryl; wherein two of R¹, R¹′, R², R²′, and R³ may be taken together to form a cycloalkyl, heterocyclo, aryl, and heteroaryl.

In an embodiment of the invention, preferred groups of compounds of Formula IV include those compounds wherein (1) A is CR′, wherein R′ is H; B is S; and D is CR′, wherein R′ is H; (2) A is NH; B is CR′, wherein R′ is H; and D is CR′, wherein R′ is H; (3) A is CR′═CH, wherein R′ is H; B is CR′, wherein R′ is H; and D is CR′, wherein R′ is H; and (4) A is CR′, wherein R′ is H; B is CR′, wherein R′ is H; and D is O.

The present invention is directed to a compound of Formula V

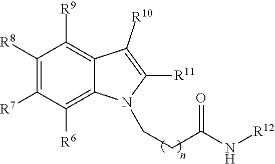

including all pharmaceutically acceptable salts, crystalline forms, stereoisomers, prodrugs, and amorphous forms, wherein R⁶, R⁷, R⁸, R⁹, R¹⁰, and R¹¹ are each individually selected from the group consisting of H, alkyl, alkenyl, alkynyl, hydroxy, alkoxy, alkenyloxy, alkynyloxy, alkylthiol, alkylsulfoxide, alkylsulfone, alkylthioester, halogen, carboxylate, amido, aryl, and heteroaryl; R¹² is selected from the group consisting of H, alkyl, alkenyl, alkynyl, alkoxy, hydroxy, alkenyloxy, alkynyloxy, alkylthiol, alkylsulfoxide, alkylsulfone, alkylthioester, halogen, carboxylic acid, carboxylate, amido, aryl, and heteroaryl; and n is an integer selected from the group consisting of 0, 1, 2, or 3.

In an embodiment of the invention, preferred groups of compounds of Formula V include those compounds wherein R¹² is a substituted alkyl group. The substituted alkyl may be substituted with an optionally substituted amido group. The optionally substituted alkyl group may be substituted with a substituent selected from the group consisting of alkyl carboxylic acid and alkyl-amido carboxylic acid.

The present invention is directed to a compound of Formula VI

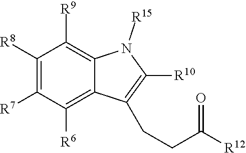

Including all pharmaceutically acceptable salts, crystalline forms, stereoisomers, prodrugs, and amorphous forms, wherein R⁶, R⁷, R⁸, R⁹, and R¹⁰ are each individually selected from the group consisting of H, alkyl, alkenyl, alkynyl, hydroxy, alkoxy, alkenyloxy, alkynyloxy, alkylthiol, alkylsulfoxide, alkylsulfone, alkylthioester, halogen, carboxylate, amido, aryl, and heteroaryl; R¹² is selected from the group consisting of H, alkyl, alkenyl, alkynyl, alkoxy, hydroxy, alkenyloxy, alkynyloxy, alkylthiol, alkylsulfoxide, alkylsulfone, alkylthioester, halogen, carboxylic acid, carboxylate, amido, aryl, and heteroaryl; R¹⁵ is selected from the group consisting of H, alkyl, alkenyl, and alkynyl; and n is an integer selected from the group consisting of 0, 1, 2, or 3.

In an embodiment of the invention, preferred groups of compounds of Formula VI include those compounds wherein R¹² is selected from the group consisting of hydroxy and optionally substituted amino. The optionally substituted amino may be substituted with an alkylaryl.

The present invention is directed to a compound of Formula VII

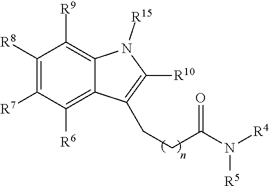

including all pharmaceutically acceptable salts, crystalline forms, stereoisomers, prodrugs, and amorphous forms, wherein R⁴ and R⁵ are each individually selected from the group consisting of H, alkyl, alkenyl, alkynyl, hydroxy, alkoxy, alkenyloxy, alkynyloxy, alkylthiol, alkylsulfoxide, alkylsulfone, alkylthioester, halogen, carboxylate, amido, aryl, and heteroaryl; R⁴ and R⁵ may be taken together to form a 5, 6, or 7 membered heterocyclo or a 5, 6, 7 membered heteroaryl; R⁶, R⁷, R⁹, R⁹, and R¹⁰ are each individually selected from the group consisting of H, alkyl, alkenyl, alkynyl, hydroxy, alkoxy, alkenyloxy, alkynyloxy, alkylthiol, alkylsulfoxide, alkylsulfone, alkylthioester, halogen, carbonyl, carboxylic acid, carboxylate, amido, aryl, and heteroaryl; R15 is selected from the group consisting of H, alkyl, alkenyl, and alkynyl; and n is an integer selected from the group consisting of 0, 1, 2, or 3.

In an embodiment of the invention, preferred groups of compounds of Formula VII include those compounds wherein the heterocyclo or heteroaryl formed by R⁴ and R⁵ may be substituted with an α, β, γ, or δ—alkylcarboxylic acid.

In another embodiment of the invention, preferred groups of compounds of Formula VI include those compounds wherein R⁴ and R⁵ taken together yield a piperazine.

The present invention is directed to a compound of Formula VIII

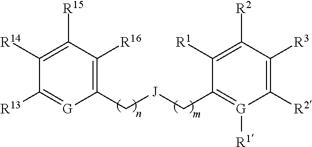

including all pharmaceutically acceptable salts, crystalline forms, stereoisomers, prodrugs, and amorphous forms, wherein G each instance is individually selected from the group consisting of CH and N; J is selected from the group consisting of CH, NR′, O, and S; R′ is selected from the group consisting of H and alkyl; R¹, R¹′, R², R²′, and R³ are each individually selected from the group consisting of H, alkyl, alkenyl, alkynyl, hydroxy, alkoxy, alkenyloxy, alkynyloxy, alkylthiol, alkylsulfoxide, alkylsulfone, alkylthioester, halogen, carbonyl, carboxylic acid, carboxylate, cyano, amido, aryl, and heteroaryl; R¹³, R¹⁴, R¹⁵, and R¹⁶ are each individually selected from the group consisting of H, alkyl, alkenyl, alkynyl, carboxylic acid, and carboxylate; and m and n are each individually selected from an integer selected from the group consisting of 0, 1, 2, or 3.

In an embodiment of the invention, preferred groups of compounds of Formula VIII include those compounds wherein m and n are each individually an integer selected from the group consisting of 0 or 1; wherein m is 0; and n is 1; or wherein m is 1; and n is 0.

The present invention is directed to a pharmaceutical composition comprising a compound of any one of Formulas 1 to VIII or an enantiomer, diastereomer, hydrate, solvate, or a pharmaceutically acceptable salt thereof, and at least one pharmaceutically acceptable carrier or excipient.

The present invention is directed to a method of inhibiting the expression and/or activity of RCF40 in a subject, comprising administering to the subject a compound according to any one of Formulas I to VIII or an enantiomer, diastereomer, hydrate, solvate, or a pharmaceutically acceptable salt thereof, wherein the activity of RCF40 activity is inhibited in the subject.

The present invention is directed to a method of ameliorating or treating RCF40-mediated cancers in a subject, comprising administering to the subject an effective amount of a compound according to any one of Formulas I to VIII or an enantiomer, diastereomer, hydrate, solvate, or a pharmaceutically acceptable salt thereof, wherein the growth of cancer cells is inhibited in a subject. The cancers may be breast cancer, kidney cancer, choriocarcinoma, acute myeloid leukemia, chronic myeloid leukemia, nasopharyngeal cancer, glioblastomas, prostate cancer, cervical cancer, ovarian cancer, uterine cancer, and hepatocellular carcinoma. The breast cancer may be estrogen sensitive breast cancer, HER2 positive breast cancer or triple negative breast cancer.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A-C. FIGS. 1A-1B are images of stained sections of 96 cores of patient breast tumor micro-arrays subjected to immunohistochemical analysis. Images of the stained sections of normal samples (FIG. 1A) and TNBC samples (FIG. 1B) were collected using Dako Cytomation system. FIG. 1C is a graph of RFC40 protein staining scores (intensity of staining×percentage of cells stained) in normal and TNBC samples. Statistical analysis was performed using Student's t test and the RFC40 staining score reported as mean SEM±. *Indicates P<0.05.

FIGS. 2A-B. FIG. 2A are images of 96 cores of patient breast tumor micro-arrays subjected to fluorescent in situ hybridization (FISH), using a probe that hybridized to the RFC40 promoter region (Red dot; white arrows) located on chromosome 7 at q11.23 position and an internal control-chromosome enumeration probe 7 (CEP7; Green dot; yellow arrows). FIG. 2B is a graph of the polysomy (green bar) and RFC40 gene amplification (red bar) observed in the total % TNBC patients as compared to normal breast tissues.

FIGS. 3A-D. FIGS.3A-3B are histograms. MCF10A cells were transiently transfected with control (GFP-Ad pDNA alone; GFP-Ad) and full-length RFC40 gene cloned in an adenoviral vector (RFC40-Ad) for 48 hr., followed by FACS analysis to determine the percentage of S-phase cells, using univariate analysis of cellular DNA content. The histograms demonstrate the percentage of S-phase cells, in MCF10A cells expressing (FIG. 3A) GFP-Ad and (FIG. 3B) RFC40-Ad. FIG. C is a Western blot of protein expression of Cyclin D1, Cyclin A and Cyclin B1 in MCF10A cells expressing GFP-Ad and RFC40-Ad. FIG. D is a Western blot of protein expression of E-cadherin and N-cadherin in MCF10A cells expressing GFP-Ad and RFC40-Ad.

FIGS. 4A-4B are graphs. FIG. 4A is a graph of the fold decrease in the cell numbers of MDA-MB-231 and MDA-MB-46 normalized to its respective control, and compared to MCF10A cells, after the MCF10A, MDA-MB-231 and MDA-MB-468 cells were transfected with non-targeting (NT)-and RFC40-siRNA. FIG. 4B is a graph of the fold decrease in the cell numbers of MDA-MB-231 normalized to its respective control, and compared to MCF10A cells, after the MCF10A and MDA-MB231 cells were transfected with miRY1005, for 72 hr. The cell numbers are reported as mean±SEM. Statistical analysis was performed using One-way ANOVA (for FIG. 4A) and Student's t test (for FIG. 4B).

FIGS. 5A-C are graphs. FIG. 5A is a graph of the effects of different concentrations of compounds labeled RDY00012, RDY00034, RDY00209, and RDY00120, on cell viability of MDA-MB-231 cells, after incubation of the drugs with the MDA-MB 231 cells for 24 hr. FIGS. 5B-5C are graphs showing selective inhibition of cell viability of MDA-MB-231 cells but not MCF10A cells by compounds labeled RDY00120, RDY00209, RDY00012 & RDY00034 after incubation of the drugs with the MDA-MB 231 cells for 24 hr.

FIG. 6 is a graph of the effects of different concentrations of compounds labelled RDY00120, RDY00209, RDY00012 and RDY00034, on induction of apoptosis in MDA-MB-231 cells after incubation with the MDA-MB-231 cells for 24 hr. The graph shows that RDY00120, RDY00209, RDY00012 & RDY00034 did not induce apoptosis in MDA-MB-231 cells.

FIG. 7 represents the KAT-Acetylation Set Enrichment-Based method (ASEB) analysis. The specific lysine residues in RFC40 that may be acetylated by CBP/p300 lysine acetyltransferases along with their respective P-values are tabulated and each of the candidate sites on RFC40 are depicted schematically. FIG. 7 discloses SEQ ID NOS: 1-4, respectively.

FIG. 8 represents the KAT-ASEB analysis. The specific lysine residues in RFC40 that may be acetylated by GNC5/PCAF lysine acetyltransferases along with their respective P-values are tabulated and each of the candidate sites on RFC40 are depicted schematically. FIG. 8 discloses SEQ ID NOS: 5-11, respectively.

FIG. 9 is a Western blot of lysed MCF10A, MCF7 and MDA-MB-231 cells and 35 μg of total protein lysates analyzed on 10% SDS-polyacrylamide gels. Protein expression of RFC40 was examined, with β-Actin used as a loading control.

FIG. 10 is a Western blot of lysates from GFP-Ad and RFC40-AD transfected MCF10cells analyzed on 10% SDS-polyacrylamide gels for expression of cGFP, RFC40 and β-Actin.

FIG. 11 represents the BDM-PUB site analysis for Ubiquitination sites on RFC40. The specific lysine residues in RFC40 that may be ubiquitinated along with their respective scores are tabulated and each of the candidate sites on RFC40 are depicted schematically. FIG. 11 discloses SEQ ID NOS: 12-19, respectively.

FIG. 12 represents the Deacetylation Site Prediction analysis. The specific lysine residues in RFC40 that may be deacetylated by HDAC1/2/3 deacetylases along with their respective P-values are tabulated and each of the candidate sites on RFC40 are depicted schematically. FIG. 12 discloses SEQ ID NOS: 20-30, respectively.

FIGS. 13A-B are images of MCF10A cells (FIG. 13A), and MDA-MB-231 cells (FIG. 13B) incubated with polyclonal anti-RFC40 and monoclonal anti-HDAC3 primary antibodies followed by incubation with Alex-488-labeled anti-rabbit and Alexa-568-labeled anti-mouse secondary antibodies, respectively. Nuclei were s counterstained with DAPI. Images were collected using an Olympus Plan x20/NA 0.25 Phi objective. In each experiment, all data were collected at identical imaging settings. RFC40=green; HDAC3=red; nucleus=blue. Yellow color indicates co-localization of RFC40 and HDAC3 in the merged images (n=4 for MDA-MB-231 cells).

FIG. 14 is a Western blot of protein expression of RFC40, with β-Actin used as loading control, in HEK293 cells incubated with two different concentrations of the compound labeled RDY00012 for 24 hr.

FIGS. 15A-B are graphs of RFC40 ATPase activity inhibition. Human recombinant RFC40 (250 nM) was pre-incubated, individually, with different concentrations of compounds labeled RDY00209 and RDY00120 at 37° C. ATP (25 μM) was then added to the mixture and the ATPase enzyme activity of RFC40 was estimated using the ADP-Glo Max Assay. FIG. 15A presents RFC40 enzyme activity after a 30 min incubation with each compound, and FIG. 15B presents RFC40 enzyme activity after a 90 min incubation with each compound.

### DETAILED DESCRIPTION OF CERTAIN EMBODIMENTS

Unrestricted proliferation, as observed in cancerous cells, requires a continuous supply of the DNA replication proteins, and therefore over-expression of replication protein(s) may be associated with deregulation of growth control, leading to malignant transformation. Additionally, cancers with gene copy number amplification or chromosome polysomy usually are the most aggressive in nature^{13, 26-28} and cannot be corrected at the chromosomal level. In such cases, the key for effective cancer treatment is early detection and the development of potent targeted drugs that will selectively target protein(s) directly involved in the cancer cell proliferation and are receptor independent. Over-expression of RFC40, which is required for different DNA transactions (DNA replication; mitosis and cytokinesis; DNA checkpoint repair; genomic stability and sister chromatid cohesion in the cell), may be instrumental in causing unrestricted proliferation, leading to cancer. Furthermore, its over-expression in tissues of varied origin is not accidental, but rather suggests that its aberrant expression confers growth advantages to cancer cells.

Since the DNA replication machinery does not change irrespective of the tissue type or extracellular stimuli coming from either cell surface, endocrine and/or growth receptors, the identification of the RFC40 molecular target involved in DNA replication offers a global treatment for a variety of cancers by targeting the most essential DNA replication machinery required for the survival of cancer cells. Since there are no reported polymorphisms for the RFC40 gene/protein, target-based therapy against this protein will cover cancer treatment across all ethnic groups. Furthermore, its over-expression in cancers of varied tissue origin, including choriocarcinoma, acute and chronic myeloid leukemia, nasopharyngeal cancer, glioblastomas, hepatocellular carcinoma, and all subtypes of breast cancer, makes it a universal molecular target for drug therapy. Additionally, since the assembly of the RFC complex is the first step in DNA elongation and commits the cell for DNA replication, over-expression of RFC40 might be an early event in the development of cancer. Thus, RFC40 provides a non-receptor diagnostic and prognostic marker and a therapeutic or interventional target involved in cell division and proliferation which, without intending to be constrained by theory, is independent of growth factor and endocrine receptor status. Inhibition or modulation of RFC40 is therefore applicable in early-stage cancer, late-stage cancer, on drug failure or resistance to receptor-based therapies, and in instances of cancer recurrence.

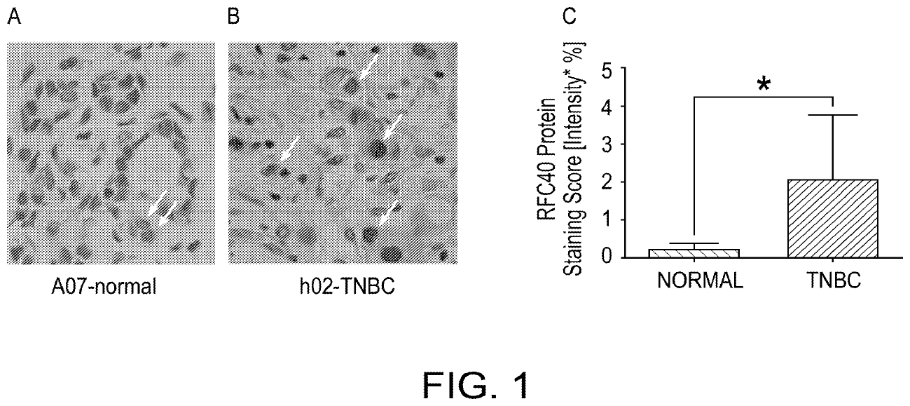

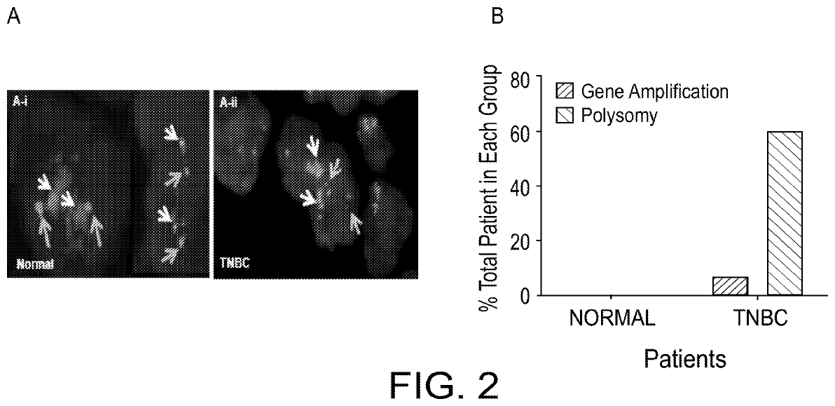

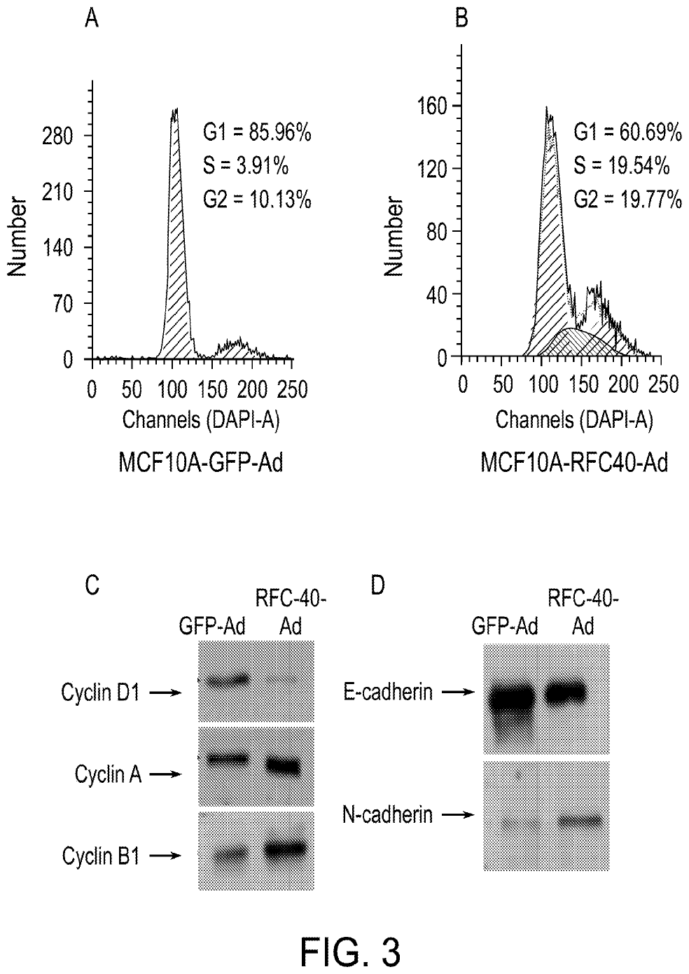

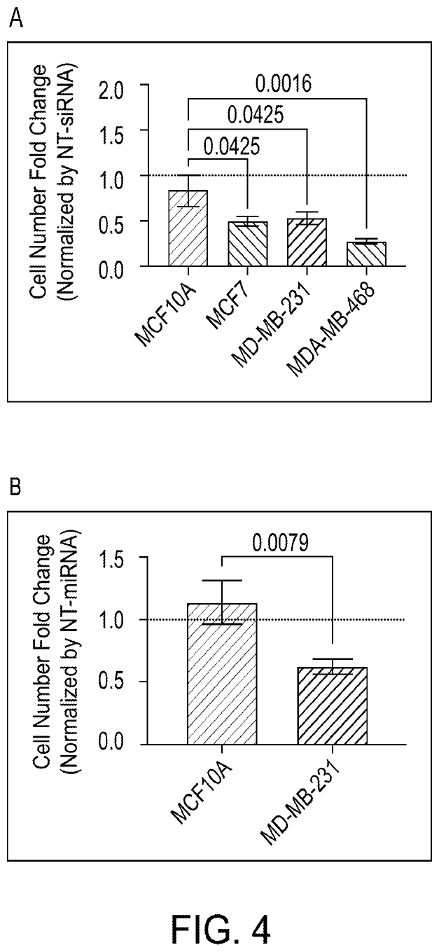

In breast cancer, it has been found that the message of RFC40 was up-regulated by 5.97-fold in TNBC cells, as compared to non-cancerous breast cells.²⁶ Furthermore, RFC40 protein was up-regulated by 8.6-fold in TNBC patient breast tumors (FIG. 1). Interestingly, RFC40 gene copy number gain was observed in 55-62% of patients with breast tumors (FIG. 2), with 40% of the patients having tumor size of 2-5 cm and in 90% of patients when the cancer had metastasized to the regional lymph node(s). Additionally, over-expression of RFC40 in non-cancerous breast epithelial cells (MCF10A), resulted in the increase in the percentage of S-phase cells from 3.91% in control to 19.54% (FIGS. 3A-3Bi), with simultaneous up-regulation of Cyclin A (S-phase marker) and Cyclin B1 (G2/M-phase marker) and down-regulation of Cyclin D1 (G1-phase marker), in the RFC40-over-expressed MCF10A cells, as compared to control MCF10A cells (FIG. 3C). This suggested that over-expression of RFC40 in MCF10A cells promoted an increase in the number of S-phase cells similar to those seen in cancerous cells. Moreover, over-expression of RFC40 in MCF10A, induced phenotypic transitions of non-cancerous epithelial breast cells, that are hallmarks of oncogenic transformations. Specifically, down-regulation of the epithelial marker proteins (e.g., E-cadherin) to over-expression of the mesenchymal proteins (e.g. N-cadherin, FIG. 3D) was observed.²⁴⁻²⁵ Conversely, knocking down endogenous RFC40, in TNBC cells, using siRNA/miRNA, resulted in selective inhibition of TNBC cells by 76%, without affecting the non-cancerous breast cells (FIG. 4A).²⁶ Furthermore, treatment of female nu/nu mice harboring a TNBC xenograft tumor with a shRNA designated miRY1005 resulted in tumor growth/size inhibition (TGI), by 65% (FIG. 4B).²³ Taken together, it can be seen that RFC40 is critical in the pathogenesis of TNBC and therefore, is a pharmacotherapeutic target for TNBC.

Importantly, RFC40-targeted therapies will offer therapeutic benefits that may potentially overcome the shortcomings of the currently available therapies. Unlike conventional chemotherapeutic drugs that globally bind to DNA directly, causing inhibition of DNA synthesis as well as DNA repair, targeting RFC40 with drug therapies would inhibit the formation of the RFC complex, thereby completely stalling all DNA related transactions, without damaging the DNA itself. This approach would provide an advantageous alternative to conventional drugs by significantly minimizing the off-target effects on DNA as well as other proteins involved in DNA repair.

The invention provides compounds for use in inhibiting the growth and/or cell proliferation of cancer cells by inhibiting the activity and/or expression of RFC40. The cancer cells whose growth is to be inhibited are cancer cells that overexpress RFC40, in particular all subtypes of breast cancer cells, choriocarcinoma, acute and chronic myeloid leukemia, nasopharyngeal cancer, glioblastomas, hepatocellular carcinoma, prostate cancer, uterine cancer, ovarian cancer, and cervical cancer. The breast cancer cells are estrogen positive, estrogen negative/HER2 positive or progesterone/estrogen/HER2 negative breast cancer cells (TNBCs). Compounds that inhibit RCF40 expression and/or activity in cancer cells and compositions comprising such compounds, include compounds of Formula I, II, III, IV, V, VI, VII, and VIII, described below. These compounds were found to inhibit cell viability of TNBC cells up to 95% without affecting non-cancerous breast cells. As demonstrated in the Examples, compounds of the present invention inhibited cell viability only in TNBC cells, thereby restricting the inhibition of RFC40 to cancerous cells only and thus offering selectivity for therapeutic intervention. Additionally, the inhibition of cell viability occurred due to necrosis/necroptosis and not apoptosis, thus providing an alternate therapy for apoptosis resistant cancer cells.

The compound of Formula I comprises:

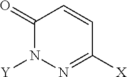

including all pharmaceutically acceptable salts, crystalline forms, stereoisomers, prodrugs, and amorphous forms, wherein
X is selected from the group consisting of aryl and heteroaryl,
   wherein each X is optionally substituted with one or more substituents selected from the group consisting of H, halogen, alkyl, hydroxy, alkoxy, alkenyloxy, alkynyloxy, amido, alkylthiol, alkylsulfoxide, alkylsulfone, alkylthioester, and heteroaryl,
   wherein two adjacent substituents on X may be taken together to form a cycloalkyl, cycloalkenyl, heterocyclyl, and heteroaryl; and
Y is alkyl
   wherein Y is optionally substituted with one or more substituents selected from the group consisting of alkyl, alkenyl, alkynyl, amido, carbonyl, carboxyl, aryl, and heteroaryl,
   wherein two adjacent substituents on Y may be taken together to form a cycloalkyl, cycloalkenyl, heterocyclyl, and heteroaryl.

The compound of Formula I may have Y substituted with one or more substituents selected from the group consisting of amido and carboxyl. The amido may be substituted with one or more substituents selected from the group consisting of alkyl carboxylic acid, aryl, and heteroaryl. The carboxyl may be a carboxylic acid group.

The compound of Formula II comprises:

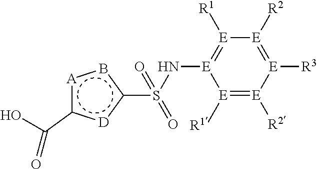

including all pharmaceutically acceptable salts, crystalline forms, stereoisomers, prodrugs, and amorphous forms, wherein
A is selected from the group consisting of CR′, CR′═CH, CR′═N, N, NH, S, and O;
B is selected from the group consisting of CR′, CR′═CH, N, NH, S, and O;
D is selected from the group consisting of CR′, N, NH, S, and O;
E at each instance is independently selected from the group consisting of C and N;
R′ at each instance is independently selected from the group consisting of H, alkyl, alkenyl, alkynyl, hydroxy, alkoxy, and halogen;
R¹ and R¹′ are each individually selected from the group consisting of H, alkyl, alkenyl, alkynyl, hydroxy, alkoxy, alkenyloxy, alkynyloxy, alkylthiol, alkylsulfoxide, alkylsulfone, alkylthioester, halogen, carboxylate, amido, aryl, and heteroaryl;
R² and R²′ are each individually selected from the group consisting of H, alkyl, alkenyl, alkynyl, hydroxy, alkoxy, alkenyloxy, alkynyloxy, alkylthiol, alkylsulfoxide, alkylsulfone, alkylthioester, halogen, carboxylate, amido, aryl, and heteroaryl; and
R³ is selected from the group consisting of H, alkyl, alkenyl, alkynyl, alkoxy, hydroxy, alkenyloxy, alkynyloxy, alkylthiol, alkylsulfoxide, alkylsulfone, alkylthioester, halogen, carboxylate, amido, aryl, and heteroaryl,
   wherein two of R¹, R¹′, R², R²′, and R³ may be taken together to form a cycloalkyl, heterocyclo, aryl, and heteroaryl.

In one embodiment of the compound of Formula II, A is CR′, wherein R′ is H; B is S; and D is CR′, wherein R′ is H.

In another embodiment of the compound of Formula II, A is NH; B is CR′, R′ is H; and D is CR′, wherein R′ is H.

In another embodiment of the compound of Formula II, A is CR′═CH, wherein R′ is H; B is CR′, wherein R′ is H; and D is CR′, wherein R′ is H.

In another embodiment of the compound of Formula II, A is CR′, wherein R′ is H; B is CR′, wherein R′ is H; and D is O.

The compound of Formula III comprises:

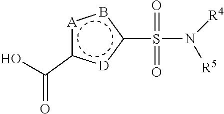

including all pharmaceutically acceptable salts, crystalline forms, stereoisomers, prodrugs, and amorphous forms, wherein
A is selected from the group consisting of CR′, CR′═CH, NH, N, S, and O;
B is selected from the group consisting of CR′, CR′═CH, NH, N, S, and O;
D is selected from the group consisting of CR′, NH, N, S, and O; and
R⁴ and R⁵ may be taken together to form a 5, 6, or 7 membered optionally substituted heterocyclo or a 5, 6, or 7 membered optionally substituted heteroaryl.

The compound of Formula IV comprises:

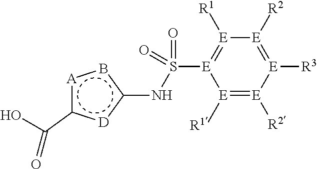

including all pharmaceutically acceptable salts, crystalline forms, stereoisomers, prodrugs, and amorphous forms, wherein
A is selected from the group consisting of CR′, CR′═CH, CR′═N, NH, N, S, and O;
B is selected from the group consisting of CR′, CR′═CH, CR′═N, NH, N, S, and O;
D is selected from the group consisting of CR′, NH, N, S, and O;
E at each instance is independently selected from the group consisting of CR′, NH and N;
R′ at each instance is independently selected from the group consisting of H, alkyl, alkenyl, alkynyl, hydroxy, alkoxy, and halogen;
R¹ and R¹′ are each individually selected from the group consisting of H, alkyl, alkenyl, alkynyl, hydroxy, alkoxy, alkenyloxy, alkynyloxy, alkylthiol, alkylsulfoxide, alkylsulfone, alkylthioester, halogen, carboxylate, amido, aryl, and heteroaryl;
R² and R²′ are each individually selected from the group consisting of H, alkyl, alkenyl, alkynyl, hydroxy, alkoxy, alkenyloxy, alkynyloxy, alkylthiol, alkylsulfoxide, alkylsulfone, alkylthioester, halogen, carboxylate, amido, aryl, and heteroaryl; and
R³ is selected from the group consisting of H, alkyl, alkenyl, alkynyl, alkoxy, hydroxy, alkenyloxy, alkynyloxy, alkylthiol, alkylsulfoxide, alkylsulfone, alkylthioester, halogen, carboxylate, amido, aryl, and heteroaryl,
   wherein two of R¹, R¹′, R², R²′, and R³ may be taken together to form a cycloalkyl, heterocyclo, aryl, and heteroaryl.

In one embodiment of the compound of Formula IV, A is CR′, wherein R′ is H; B is S; and D is CR′, wherein R′ is H.

In another embodiment of the compound of Formula IV, A is NH; B is CR′, wherein R′ is H; and D is CR′, wherein R′ is H.

In another embodiment of the compound of Formula IV, A is CR′═CH, wherein R′ is H; B is CR′, wherein R′ is H; and D is CR′, wherein R′ is H.

In another embodiment of the compound of Formula IV, A is CR′, wherein R′ is H; B is CR′, wherein R′ is H; and D is O.

The compound of Formula V comprises:

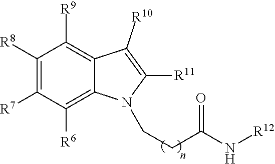

including all pharmaceutically acceptable salts, crystalline forms, stereoisomers, prodrugs, and amorphous forms, wherein
R⁶, R⁷, R⁸, R⁹, R¹⁰, and R¹¹ are each individually selected from the group consisting of H, alkyl, alkenyl, alkynyl, hydroxy, alkoxy, alkenyloxy, alkynyloxy, alkylthiol, alkylsulfoxide, alkylsulfone, alkylthioester, halogen, carboxylate, amido, aryl, and heteroaryl;
R¹² is selected from the group consisting of H, alkyl, alkenyl, alkynyl, alkoxy, hydroxy, alkenyloxy, alkynyloxy, alkylthiol, alkylsulfoxide, alkylsulfone, alkylthioester, halogen, carboxylic acid, carboxylate, amido, aryl, and heteroaryl; and
n is an integer selected from the group consisting of 0, 1, 2, or 3.

In one embodiment of the compound of Formula V, R¹² is a substituted alkyl group.

In another embodiment of the compound of Formula V, the substituted alkyl is substituted with an optionally substituted amido group.

In another embodiment of the compound of Formula V, the optionally substituted alkyl group is substituted with a substituent selected from the group consisting of alkyl carboxylic acid and alkyl-amido carboxylic acid.

The compound of Formula VI comprises:

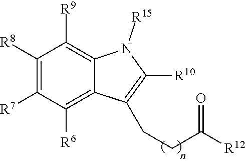

including all pharmaceutically acceptable salts, crystalline forms, stereoisomers, prodrugs, and amorphous forms, wherein
R⁶, R⁷, R⁸, R⁹, and R¹⁰ are each individually selected from the group consisting of H, alkyl, alkenyl, alkynyl, hydroxy, alkoxy, alkenyloxy, alkynyloxy, alkylthiol, alkylsulfoxide, alkylsulfone, alkylthioester, halogen, carboxylate, amido, aryl, and heteroaryl;
R¹² is selected from the group consisting of H, alkyl, alkenyl, alkynyl, alkoxy, hydroxy, alkenyloxy, alkynyloxy, alkylthiol, alkylsulfoxide, alkylsulfone, alkylthioester, halogen, carboxylic acid, carboxylate, amido, aryl, and heteroaryl;
R¹⁵ is selected from the group consisting of H, alkyl, alkenyl, and alkynyl; and
n is an integer selected from the group consisting of 0, 1, 2, or 3.

In one embodiment of the compound of Formula VI, R¹² is selected from the group consisting of hydroxy and optionally substituted amino.

In another embodiment of the compound of Formula VI, the optionally substituted amino is substituted with an alkylaryl.

The compound of Formula VII comprises:

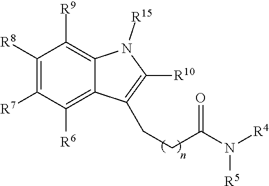

including all pharmaceutically acceptable salts, crystalline forms, stereoisomers, prodrugs, and amorphous forms, wherein
R⁴ and R⁵ are each individually selected from the group consisting of H, alkyl, alkenyl, alkynyl, hydroxy, alkoxy, alkenyloxy, alkynyloxy, alkylthiol, alkylsulfoxide, alkylsulfone, alkylthioester, halogen, carboxylate, amido, aryl, and heteroaryl;
R⁴ and R⁵ may be taken together to form a 5, 6, or 7 membered heterocyclo or a 5, 6, 7 membered heteroaryl;
R⁶, R⁷, R⁹, R⁹, and R¹⁰ are each individually selected from the group consisting of H, alkyl, alkenyl, alkynyl, hydroxy, alkoxy, alkenyloxy, alkynyloxy, alkylthiol, alkylsulfoxide, alkylsulfone, alkylthioester, halogen, carbonyl, carboxylic acid, carboxylate, amido, aryl, and heteroaryl;
R¹⁵ is selected from the group consisting of H, alkyl, alkenyl, and alkynyl; and
n is an integer selected from the group consisting of 0, 1, 2, or 3.

In one embodiment of the compound of Formula VII, heterocyclo or heteroaryl formed by R⁴ and R⁵ may be substituted with an α, β, γ, or δ—alkylcarboxylic acid.

In another embodiment of the compound of Formula VII, R⁴ and R⁵ taken together yield a piperazine.

The compound of Formula VIII comprises:

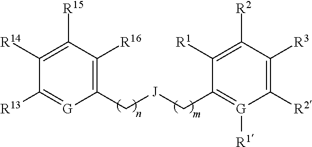

including all pharmaceutically acceptable salts, crystalline forms, stereoisomers, prodrugs, and amorphous forms, wherein
G each instance is individually selected from the group consisting of CH and N;
J is selected from the group consisting of CH, NR′, O, and S;
   R′ is selected from the group consisting of H and alkyl;
   R¹, R¹′, R², R²′, and R³ are each individually selected from the group consisting of H, alkyl, alkenyl, alkynyl, hydroxy, alkoxy, alkenyloxy, alkynyloxy, alkylthiol, alkylsulfoxide, alkylsulfone, alkylthioester, halogen, carbonyl, carboxylic acid, carboxylate, cyano, amido, aryl, and heteroaryl;
   R¹³, R¹⁴, R¹⁵, and R¹⁶ are each individually selected from the group consisting of H, alkyl, alkenyl, alkynyl, carboxylic acid, and carboxylate; and
   m and n are each individually selected from an integer selected from the group consisting of 0, 1, 2, or 3.

In one embodiment of the compound of Formula VIII, m and n are each individually an integer selected from the group consisting of 0 or 1.

In another embodiment of the compound of Formula VIII, m is 0; and n is 1.

In another embodiment of the compound of Formula VIII, m is 1; and n is 0.

Any of the foregoing compounds or an enantiomer, diastereomer, hydrate, solvate, or a pharmaceutically acceptable salt thereof can be in the form of a pharmaceutical composition, which may comprise a therapeutically effective amount of the compound and a pharmaceutically acceptable carrier or excipient. Preferred methods of administration of the compound and compositions for use in the methods of the invention are oral, intrathecal, intratumoral (breast pad), transdermal and parental including intravenous. Further methods of administration may include subcutaneous, intra-muscular, intraperitoneal, or intravesicular administration to a subject. The compound must be in the appropriate form for administration of choice.

The phrase “pharmaceutically acceptable” or “pharmacologically acceptable” as used herein refers to molecular entities and compositions that are physiologically tolerable and do not typically produce an allergic or similar untoward reaction, such as gastric upset, dizziness and the like, when administered to a human, and approved by a regulatory agency of the Federal or a state government or listed in the U.S. Pharmacopeia or other generally recognized pharmacopeia for use in animals, and more particularly in humans.

The term “carrier” refers to a diluent, adjuvant, excipient, or vehicle with which the therapeutic is administered. Such pharmaceutical carriers can be sterile liquids, such as saline solutions in water and oils, including those of petroleum, animal, vegetable, or synthetic origin, such as peanut oil, soybean oil, mineral oil, sesame oil, and the like. A saline solution is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol, and the like. The composition, if desired, can also contain minor amounts of wetting or emulsifying agents, or pH buffering agents. The use of solvents, dispersion media, coatings, antibacterial and antifungal agents, isotonic and absorption delaying agents, and the like for pharmaceutical active substances that are pharmaceutically acceptable as the term is used herein are well known in the art and are preferably inert. Except insofar as any conventional media or agent is incompatible with the active ingredients, its use in therapeutic compositions is contemplated.

Pharmaceutical compositions adapted for oral administration may be capsules, tablets, powders, granules, solutions, syrups, suspensions (in non-aqueous or aqueous liquids), or emulsions. Tablets or hard gelatin capsules may comprise lactose, starch, or derivatives thereof, magnesium stearate, sodium saccharine, cellulose, magnesium carbonate, stearic acid, or salts thereof. Soft gelatin capsules may comprise vegetable oils, waxes, fats, semi-solid, or liquid polyols. Solutions and syrups may comprise water, polyols, and sugars. An active agent intended for oral administration may be coated with or admixed with a material that delays disintegration and/or absorption of the active agent in the gastrointestinal tract. Thus, the sustained release may be achieved over many hours and if necessary, the active agent can be protected from degradation within the stomach. Pharmaceutical compositions for oral administration may be formulated to facilitate release of an active agent at a particular gastrointestinal location due to specific pH or enzymatic conditions.

Oral lipid-based drug delivery systems in which a drug is encapsulated or solubilized in lipid excipients can be used to increase solubilization and absorption of a drug, such as a poorly water-soluble drug, to obtain enhanced bioavailability. Various lipid excipients and formulation approaches are described in Kalepu, S. et al., Acta Pharmaceutica Sinica B 2013, 3(6):361-372.

A further preferred form of administration is parenteral including intravenous administration. Pharmaceutical compositions adapted for parenteral administration, including intravenous administration, include aqueous and non-aqueous sterile injectable solutions or suspensions, which may contain antioxidants, buffers, bacteriostats, and solutes that render the compositions substantially isotonic with the blood of the subject. Other components which may be present in such compositions include water, alcohols, polyols, glycerin, and vegetable oils. Compositions adapted for parental administration may be presented in unit-dose or multi-dose containers, such as sealed ampules and vials, and may be stored in a freeze-dried (lyophilized) condition requiring only the addition of a sterile carrier, immediately prior to use. Extemporaneous injection solutions and suspensions may be prepared from sterile powders, granules, and tablets. Suitable vehicles that can be used to provide parenteral dosage forms of the invention are well known to those skilled in the art. Examples include: Water for Injection USP; aqueous vehicles such as Sodium Chloride Injection, Ringer's Injection, Dextrose Injection, Dextrose and Sodium Chloride Injection, and Lactated Ringer's Injection; water-miscible vehicles such as ethyl alcohol, polyethylene glycol, and polypropylene glycol; and non-aqueous vehicles such as corn oil, cottonseed oil, peanut oil, sesame oil, ethyl oleate, isopropyl myristate, and benzyl benzoate.

Any of the foregoing compounds or an enantiomer, diastereomer, hydrate, solvate, or a pharmaceutically acceptable salt thereof can be used to inhibit the expression and/or activity of RCF40 in a subject, comprising administering to the subject the compound or an enantiomer, diastereomer, hydrate, solvate, or a pharmaceutically acceptable salt thereof.

Any of the foregoing compounds or an enantiomer, diastereomer, hydrate, solvate, or a pharmaceutically acceptable salt thereof can be used to ameliorate or treat RCF40-mediated cancers in a subject, comprising administering to the subject an effective amount of the compound or an enantiomer, diastereomer, hydrate, solvate, or a pharmaceutically acceptable salt thereof, and the growth of cancer cells is inhibited in the subject. The cancers to be treated include, but are not limited to, breast cancer, kidney cancer, choriocarcinoma, acute myeloid leukemia, chronic myeloid leukemia, nasopharyngeal cancer, glioblastomas, prostate cancer, cervical cancer, ovarian cancer, uterine cancer, and hepatocellular carcinoma. The breast cancer can be estrogen sensitive breast cancer, HER2 positive breast cancer or triple negative breast cancer.

Selection of a therapeutically effective dose will be determined by the skilled artisan considering several factors, which will be known to one of ordinary skill in the art. Such factors include the particular form of the pharmacological agent, and its pharmacokinetic parameters such as bioavailability, metabolism, and half-life, which will have been established during the usual development procedures typically employed in obtaining regulatory approval for a pharmaceutical compound. Further factors in considering the dose include the condition or disease to be treated or the benefit to be achieved in a normal individual, the body mass of the patient, the route of administration, whether the administration is acute or chronic, concomitant medications, and other factors well known to affect the efficacy of administered pharmaceutical agents. Thus, the precise dose should be decided according to the judgment of the person of skill in the art, and each patient's circumstances, and according to standard clinical techniques.

### Definitions

The terms used in this specification generally have their ordinary meanings in the art, within the context of this invention and the specific context where each term is used. Certain terms are discussed below, or elsewhere in the specification, to provide additional guidance to the practitioner in describing the methods of the invention and how to use them. Moreover, it will be appreciated that the same thing can be said in more than one way. Consequently, alternative language and synonyms may be used for any one or more of the terms discussed herein, nor is any special significance to be placed upon whether or not a term is elaborated or discussed herein. Synonyms for certain terms are provided. A recital of one or more synonyms does not exclude the use of the other synonyms. The use of examples anywhere in the specification, including examples of any terms discussed herein, is illustrative only, and in no way limits the scope and meaning of the invention or any exemplified term. Likewise, the invention is not limited to its preferred embodiments.

The term “subject” or “patient: as used in this application means an animal with an immune system such as avians and mammals. Mammals include humans, canines, felines, rodents, bovine, equines, porcines, ovines, and primates. Avians include, but are not limited to, fowls, songbirds, and raptors. Thus, the invention can be used in human medicine and in veterinary medicine, e.g., to treat companion animals, farm animals, laboratory animals in zoological parks, and animals in the wild. The invention is particularly desirable for human medical applications.

The term “in need thereof” would be a subject known or suspected of having or being at risk of cancer.

The terms “treat”, “treating” or “treatment” of a state, disorder or condition includes: (1) preventing or delaying the appearance of clinical symptoms of the state, disorder, or condition developing in a person who may be afflicted with or predisposed to the state, disorder or condition but does not yet experience or display clinical symptoms of the state, disorder or condition; or (2) inhibiting the state, disorder or condition, i.e., arresting, reducing or delaying the development of the disease or a relapse thereof (in case of maintenance treatment) or at least one clinical symptom, sign, or test, thereof; or (3) relieving the disease, i.e., causing regression of the state, disorder or condition or at least one of its clinical or sub-clinical symptoms or signs.

The term “agent” as used herein means a substance that produces or is capable of producing an effect and would include, but is not limited to, chemicals, pharmaceuticals, biologics, small organic molecules, antibodies, nucleic acids, peptides, and proteins. The terms “agent”, “compound” and “drug” are interchangeable.

The use of the singular herein includes the plural (and vice versa) unless specifically stated otherwise.

The term “about” or “approximately” means within an acceptable error range for the particular value as determined by one of ordinary skill in the art, which will depend in part on how the value is measured or determined, i.e., the limitations of the measurement system, i.e., the degree of precision required for a particular purpose, such as a pharmaceutical formulation. For example, “about” can mean within 1 or more than 1 standard deviations, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value. Where particular values are described in the application and claims, unless otherwise stated, the term “about” meaning within an acceptable error range for the particular value should be assumed.

As used herein, the terms “therapeutically effective amount”, “therapeutically effective dose” and “effective amount” refer to an amount of the compound and compositions which is sufficient to effect beneficial or desired results, that, when administered alone or in combination with an additional therapeutic agent to a cell, tissue, or subject, is effective to inhibit the activity and/or expression of RCF40 in cancer cells. A therapeutically effective dose further refers to that amount of the compound sufficient to result in at least partial amelioration of symptoms, e.g., treatment, healing, prevention or amelioration of the relevant medical condition, or an increase in rate of treatment, healing, prevention, or amelioration of such conditions. When applied to an individual active ingredient administered alone, a therapeutically effective dose refers to that ingredient alone. When applied to a combination, a therapeutically effective dose refers to combined amounts of the active ingredients that result in the therapeutic effect, whether administered in combination, serially or simultaneously. The effective amount of the compound may be less when administered in a combination than when administered alone. An effective amount can also result in an improvement in a subjective measure in cases where subjective measures are used to assess disease severity.

The term “selective inhibition” or “selectively inhibit” refers to the ability of a biologically active agent to preferentially inhibit cancer cells as compared to non-cancerous cells such as, e.g., non-cancerous breast cells, by alleviating the activity and/or expression of RFC40 in cancer cells via direct or indirect interaction with the target.

The term “pharmaceutical composition” means a mixture of substances suitable for administering to a subject. Pharmaceutical compositions can comprise, for example, a combination of pharmaceutical agents as well as the presence of a sterile aqueous solution or other standard pharmaceutical additive known in the art.

The term “administering” means providing a pharmaceutical agent or composition to a subject, and includes, but is not limited to administering by a medical professional and self-administering. Co-administration is the administration of two or more pharmaceutical agents to a subject. The two or more pharmaceutical agents can be in a single pharmaceutical composition or can be in separate pharmaceutical compositions. Each of the two or more pharmaceutical agents can be administered through the same or different routes of administration. Co-administration encompasses administration in parallel, concomitant or sequentially.

Throughout the description, where compositions are described as having, including, or comprising specific components, it is contemplated that compositions of the present teachings also consist essentially of, or consist of, the recited components.

In the application, where an element or component is said to be included in and/or selected from a list of recited elements or components, it should be understood that the element or component can be any one of the recited elements or components and can be selected from a group consisting of two or more of the recited elements or components.

It should be understood that the order of steps or order for performing certain actions is immaterial so long as the present teachings remain operable. Moreover, two or more steps or actions can be conducted simultaneously.

The term “halogen” shall mean chlorine, bromine, fluorine, and iodine.

Unless otherwise noted, the term “alkyl” and/or “aliphatic” whether used alone or as part of a substituent group refers to straight and branched carbon chains having 1 to 20 carbon atoms or any number within this range, for example, 1 to 6 carbon atoms or 1 to 4 carbon atoms. Designated numbers of carbon atoms (e.g., C₁₋₆) shall refer independently to the number of carbon atoms in an alkyl moiety or to the alkyl portion of a larger alkyl-containing substituent. Non-limiting examples of alkyl groups include methyl, ethyl, n-propyl, iso-propyl, n-butyl, sec-butyl, iso-butyl, tert-butyl, and the like. Alkyl groups can be optionally substituted. Non-limiting examples of substituted alkyl groups include hydroxymethyl, chloromethyl, trifluoromethyl, aminomethyl, 1-chloroethyl, 2-hydroxyethyl, 1,2-difluoroethyl, 3-carboxypropyl, and the like. In substituent groups with multiple alkyl groups such as (C₁₋₆ alkyl)₂amino, the alkyl groups may be the same or different.

The terms “alkenyl” and “alkynyl” groups, whether used alone or as part of a substituent group, refer to straight and branched carbon chains having 2 or more carbon atoms, preferably 2 to 20, wherein an alkenyl chain has at least one double bond in the chain and an alkynyl chain has at least one triple bond in the chain. Alkenyl and alkynyl groups can be optionally substituted. Nonlimiting examples of alkenyl groups include ethenyl, 3-propenyl, 1-propenyl (also 2-methylethenyl), isopropenyl (also 2-methylethen-2-yl), buten-4-yl, and the like. Nonlimiting examples of substituted alkenyl groups include 2-chloroethenyl (also 2-chlorovinyl), 4-hydroxybuten-1-yl, 7-hydroxy-7-methyloct-4-en-2-yl, 7-hydroxy-7-methyloct-3,5-dien-2-yl, and the like. Nonlimiting examples of alkynyl groups include ethynyl, prop-2-yn-1-yl (also propargyl), propyn-1-yl, and 2-methyl-hex-4-yn-1-yl. Nonlimiting examples of substituted alkynyl groups include, 5-hydroxy-5-methylhex-3-ynyl, 6-hydroxy-6-methylhept-3-yn-2-yl, 5-hydroxy-5-ethylhept-3-ynyl, and the like.

The term “cycloalkyl,” whether used alone or as part of another group, refers to a non-aromatic carbon-containing ring including cyclized alkyl, alkenyl, and alkynyl groups, e.g., having from 3 to 14 ring carbon atoms, preferably from 3 to 7 or 3 to 6 ring carbon atoms, or even 3 to 4 ring carbon atoms, and optionally containing one or more (e.g., 1, 2, or 3) double or triple bond. Cycloalkyl groups can be monocyclic (e.g., cyclohexyl) or polycyclic (e.g., containing fused, bridged, and/or spiro ring systems), wherein the carbon atoms are located inside or outside of the ring system. Any suitable ring position of the cycloalkyl group can be covalently linked to the defined chemical structure. Cycloalkyl rings can be optionally substituted. Nonlimiting examples of cycloalkyl groups include: cyclopropyl, 2-methyl-cyclopropyl, cyclopropenyl, cyclobutyl, 2,3-dihydroxycyclobutyl, cyclobutenyl, cyclopentyl, cyclopentenyl, cyclopentadienyl, cyclohexyl, cyclohexenyl, cycloheptyl, cyclooctanyl, decalinyl, 2,5-dimethylcyclopentyl, 3,5-dichlorocyclohexyl, 4-hydroxycyclohexyl, 3,3,5-trimethylcyclohex-1-yl, octahydropentalenyl, octahydro-1 H-indenyl, 3a,4,5,6,7,7a-hexahydro-3H-inden-4-yl, decahydroazulenyl; bicyclo[6.2.0]decanyl, decahydronaphthalenyl, and dodecahydro-1 H-fluorenyl. The term “cycloalkyl” also includes carbocyclic rings which are bicyclic hydrocarbon rings, non-limiting examples of which include, bicyclo-[2.1.1]hexanyl, bicyclo[2.2.1]heptanyl, bicyclo[3.1.1]heptanyl, 1,3-dimethyl[2.2.1]heptan-2-yl, bicyclo[2.2.2]octanyl, and bicyclo[3.3.3]undecanyl.

The term “alkoxy” refers to the group —O-alkyl, wherein the alkyl group is as defined above. Alkoxy groups optionally may be substituted. Examples of alkoxy groups include but are not limited to, ethoxy, isopropoxy and trifluoromethoxy groups.

The term “cycloalkenyl”, whether used alone or as part of another group, refers to a non-aromatic carbon-containing ring having at least three or more carbon atoms linked together with at least one carbon-carbon double bond to form a structural ring. Any suitable ring position of the cycloalkenyl group can be covalently linked to the defined chemical structure.

The term “alkenyloxy” or “alkynyloxy”, refers to the group —O-alkenyl or —O-alkynyl, wherein the alkenyl group and the alkynyl group are as defined above. Alkenyloxy and alkynyloxy groups optionally may be substituted.

The term “alkylthiol” refers to the group alkyl—SH, wherein the alkyl group is defined above. It is well understood in the art that an alkylthiol group may be readily oxidized to yield a “sulfoxide”, “sulfone”, or “thioester”.

The term “carbonyl” refers to a divalent chemical unit consisting of a carbon and an oxygen connect by a double bond.

The term “carboxylic acid” refers to an organic acid that contains a carboxyl group (C(═O)OH) attached to an R-group. The general formula of a carboxylic acid is R—COOH or R—CO₂H, with R referring to an alkyl, alkenyl, aryl, or other group.

The term “carboxylate” refers to a salt or ester of a carboxylic acid.

The term “aryl,” wherein used alone or as part of another group, is defined herein as an unsaturated, aromatic monocyclic ring of 6 carbon members or an unsaturated, aromatic polycyclic ring of from 10 to 14 carbon members. Aryl rings can be, for example, phenyl or naphthyl ring each optionally substituted with one or more moieties capable of replacing one or more hydrogen atoms. Non-limiting examples of aryl groups include: phenyl, naphthylen-1-yl, naphthylen-2-yl, 4-fluorophenyl, 2-hydroxyphenyl, 3-methylphenyl, 2-amino-4-fluorophenyl, 2-(N,N-diethylamino)phenyl, 2-cyanophenyl, 2,6-di-tert-butylphenyl, 3-methoxyphenyl, 8-hydroxynaphthylen-2-yl 4,5-dimethoxynaphthylen-1-yl, and 6-cyano-naphthylen-1-yl. Aryl groups also include, for example, phenyl or naphthyl rings fused with one or more saturated or partially saturated carbon rings (e.g., bicyclo[4.2.0]octa-1,3,5-trienyl, indanyl), which can be substituted at one or more carbon atoms of the aromatic and/or saturated or partially saturated rings.

The terms “heterocyclic” and/or “heterocycle” and/or “heterocyclyl,” and/or “heterocyclo” whether used alone or as part of another group, are defined herein as one or more ring having from 3 to 20 atoms wherein at least one atom in at least one ring is a heteroatom selected from nitrogen (N), oxygen (O), or sulfur (S), and wherein further the ring that includes the heteroatom is non-aromatic. In heterocycle groups that include 2 or more fused rings, the non-heteroatom bearing ring may be aryl (e.g., indolinyl, tetrahydroquinolinyl, chromanyl). One or more N or S atoms in a heterocycle group can be oxidized to yield a compound such as, for example, thiophene-1-oxide, thiophene-1,1-dioxide, pyrrolidine-n-oxide, tetrahydrothiopyran-1-oxide, tetrahydrothiopyran-1,1-dioxide, and piperidine-oxide. Heterocycle groups can be optionally substituted.

The term “heteroaryl,” whether used alone or as part of another group, is defined herein as one or more rings having from 5 to 20 atoms wherein at least one atom in at least one ring is a heteroatom chosen from nitrogen (N), oxygen (O), or sulfur (S), and wherein further at least one of the rings that includes a heteroatom is aromatic. In heteroaryl groups that include 2 or more fused rings, the non-heteroatom bearing ring may be a carbocycle or aryl. One or more N or S atoms in a heteroaryl group can be oxidized to yield a compound such as, for example, N-pyridine oxide and thiole-1-oxide. Heteroaryl groups can be substituted.

For the purposed of the present invention fused ring units, as well as spirocyclic rings, bicyclic rings and the like, which comprise a single heteroatom will be considered to belong to the cyclic family corresponding to the heteroatom containing ring.

Whenever a term or either of their prefix roots appears in a name of a substituent, the name is to be interpreted as including those limitations provided herein. For example, whenever the term “alkyl” or “aryl” or either of their prefix roots appear in a name of a substituent (e.g., arylalkyl, alkylamino) the name is to be interpreted as including those limitations given above for “alkyl” and “aryl.”

The term “substituted” is defined herein as a moiety, whether acyclic or cyclic, which has one or more hydrogen atoms replaced by a substituent or several (e.g., 1 to 10) substituents as defined herein below. The substituents are capable of replacing one or two hydrogen atoms of a single moiety at a time. In addition, these substituents can replace two hydrogen atoms on two adjacent carbons to form said substituent, new moiety, or unit. For example, a substituted unit that requires a single hydrogen atom replacement includes halogen, hydroxyl, and the like. A two hydrogen atom replacement includes carbonyl, and the like. The term “substituted” is used throughout the present specification to indicate that a moiety can have one or more of the hydrogen atoms replaced by a substituent. When a moiety is described as “substituted” any number of the hydrogen atoms may be replaced.

The variable groups defined herein, e.g., alkyl, alkenyl, alkynyl, cycloalkyl, alkoxy, alkenyloxy, alkynyloxy, aryl, heterocycle and heteroaryl groups defined herein, whether used alone or as part of another group, can be optionally substituted. Optionally substituted groups will be so indicated.

For the purposes of the present invention, the terms “compound,” “analog,” and “composition of matter” stand equally well for the compounds inhibiting the activity and/or expression of RCF40 described herein, including all enantiomeric forms, diastereomeric forms, salts, and the like, and the terms “compound,” “analog,” and “composition of matter” are used interchangeably throughout the present specification.

The term “derivative” may refer to any derivative of the present compounds. In certain embodiments, a derivative of a compound is a chemical substance related structurally to the compound and theoretically derivable from it. In certain embodiments, a derivative of a compound is a substance that can be made from the compound. In certain embodiments, a derivative of a compound is a variant of the compound. In certain embodiments, a derivative of a compound is an analog of the compound. In certain embodiments, a derivative of a compound is a chemical analog of the compound. In certain embodiments, an analog of a compound is a substance that is structurally similar to the compound but differs slightly in composition (e.g., as in the replacement of one atom by an atom of a different element or in the presence of a particular functional group). In certain embodiments, the derivative is a compound (e.g., a drug precursor or a prodrug) that is transformed in vivo to yield the present agent or a pharmaceutically acceptable salt, hydrate or solvate of the compound. The transformation may occur by various mechanisms (e.g., by metabolic or chemical processes), such as, for example, through hydrolysis in blood. Bioreversible Carriers in Drug Design, ed. Edward B. Roche, American Pharmaceutical Association and Pergamon Press, 1987.

Compounds described herein can contain an asymmetric atom (also referred as a chiral center), and some of the compounds can contain one or more asymmetric atoms or centers, which can thus give rise to optical isomers (enantiomers) and diastereomers. The present teachings and compounds disclosed herein include such enantiomers and diastereomers, as well as the racemic and resolved, enantiomerically pure R and S stereoisomers, as well as other mixtures of the R and S stereoisomers and pharmaceutically acceptable salts thereof. Optical isomers can be obtained in pure form by standard procedures known to those skilled in the art, which include, but are not limited to, diastereomeric salt formation, kinetic resolution, chiral separation by HPLC, simulated moving bed chromatography (SMB), and asymmetric synthesis. The present teachings also encompass cis and trans isomers (Z and E) of compounds containing alkenyl moieties (e.g., alkenes and imines). It is also understood that the present teachings encompass all possible regioisomers, and mixtures thereof, which can be obtained in pure form by standard separation procedures known to those skilled in the art, and include, but are not limited to, column chromatography, thin-layer chromatography, and high-performance liquid chromatography.

Pharmaceutically acceptable salts of compounds of the present teachings, which can have an acidic moiety, can be formed using organic and inorganic bases. Both mono and polyanionic salts are contemplated, depending on the number of acidic hydrogens available for deprotonation. Suitable salts formed with bases include metal salts, such as alkali metal or alkaline earth metal salts, for example sodium, potassium, or magnesium salts; ammonia salts and organic amine salts, such as those formed with morpholine, thiomorpholine, piperidine, pyrrolidine, a mono-, di- or tri-lower alkylamine (e.g., ethyl-tert-butyl-, diethyl-, diisopropyl-, triethyl-, tributyl- or dimethylpropylamine), or a mono-, di-, or trihydroxy lower alkylamine (e.g., mono-, di- or triethanolamine). Specific non-limiting examples of inorganic bases include NaHCO₃, Na₂CO₃, KHCO₃, K₂CO₃, Cs₂CO₃, LiOH, NaOH, KOH, NaH₂PO₄, Na₂HPO₄, and Na₃PO₄. Internal salts also can be formed. Similarly, when a compound disclosed herein contains a basic moiety, salts can be formed using organic and inorganic acids. For example, salts can be formed from the following acids: acetic, propionic, lactic, benzenesulfonic, benzoic, camphorsulfonic, citric, tartaric, succinic, dichloroacetic, ethenesulfonic, formic, fumaric, gluconic, glutamic, hippuric, hydrobromic, hydrochloric, isethionic, lactic, maleic, malic, malonic, mandelic, methanesulfonic, mucic, napthalenesulfonic, nitric, oxalic, pamoic, pantothenic, phosphoric, phthalic, propionic, succinic, sulfuric, tartaric, toluenesulfonic, and camphorsulfonic as well as other known pharmaceutically acceptable acids.

When any variable occurs more than one time in any constituent or in any formula, its definition in each occurrence is independent of its definition at every other occurrence. Combinations of substituents and/or variables are permissible only if such combinations result in stable compounds.

### EXAMPLES

The present invention may be better understood by reference to the following non-limiting examples, which are presented in order to more fully illustrate the preferred embodiments of the invention. They should in no way be construed to limit the broad scope of the invention.

### Example 1

### CADD Analyses Background

Computer-Aided-Drug Discovery (CADD) analyses was performed, using a RFC40 homology model and identified a custom library of small molecule entities (SMEs)/compounds. SMEs were selected, with each one being a representative of these different series based on the Extra Precision Glide (XP) score and the molecular mechanics energies combined with the generalized Born and surface area continuum solvation (MM/GBSA) score²⁴, that qualified them to have pharmaceutical grade potential with minimal noise (non-specifically binding compounds). Previous studies of identifying lead compounds, using CADD analyses, have led to the successful discovery of 12 small molecules that were approved for therapeutic use,²⁵ providing support for the expectation that these lead compounds will also advance to the clinic.

The SEM/compounds identified from the CADD analyses and their XP and MM/GBSA scores are presented in Table 1 below.

TABLE 1

| | | | MM/GBSA |
|---|---|---|---|
| Series | SME Identifier | XP Score | Score |
| 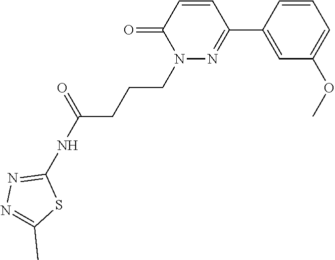 [SMILES: COc1cccc(-c2ccc(=O)n(CCCC(=O)Nc3nnc(C)s3)n2)c1] | RDY00034 | −8.207 | −97.7887 |
| Pyridazones | | | |
| 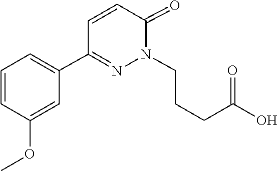 [SMILES: COc1cccc(-c2ccc(=O)n(CCCC(=O)O)n2)c1] | RDY00009 | −10.466 | −90.7918 |
| Pyridazones | | | |
| 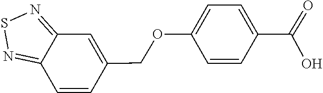 [SMILES: O=C(O)c1ccc(OCc2ccc3nsnc3c2)cc1] | RDY00012 | −8.729 | −89.1052 |
| Benzoic acids | | | |
| 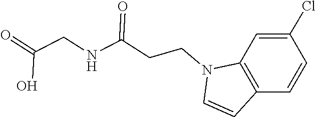 [SMILES: O=C(O)CNC(=O)CCn1ccc2ccc(Cl)cc21] | RDY00209 | −10.549 | −88.0003 |
| Indoles | | | |
| 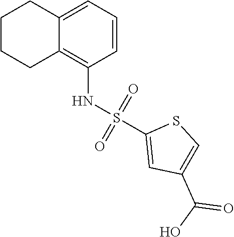 [SMILES: O=C(O)c1csc(S(=O)(=O)Nc2cccc3c2CCCC3)c1] | RDY00120 | −11.23 | −83.6901 |
| Sulfonamides | | | |

Cell-based and biochemical assays were conducted to determine whether the SMEs were biologically active. The SMEs were purchased from Mcule (Palo Alto, CA, USA). The purity of the SMEs were confirmed by LC/MS/MS and 98% pure compounds were used in the in vitro assays presented in the following examples.

### Example 2

### Cell Viability & Apoptosis

### Method

Non-cancerous breast epithelial cells (MCF10A) and TNBC cell line (MDA-MB-231; mesenchymal; originating from a Caucasian female) were purchased from the ATCC (American Type Culture Collection, Manassas, Virginia). The effect of the 7 SMEs on cell viability and apoptosis, in MDA-MB-231 and MCF10A cell lines, respectively, was determined as follows. The MCF10A and MDA-MB-231 cells were incubated with different concentrations (100 μM, 10 μM, 1 μM, 0.1 μM, 10 nM, 1 nM, 0.1 nM, and DMSO only) of the 7 SMEs for 24 hours at 37° C. Cell viability and apoptosis was estimated using the ApoLive Glo Multiplex Assay kit 29 (Promega, Madison, WI). Live-cell protease fluorescence (cell viability) was measured at 400 Eₓ/505 Eₘ and caspase3/7 activation (apoptosis) was measured by luminescence on Envision plate reader.

### Results

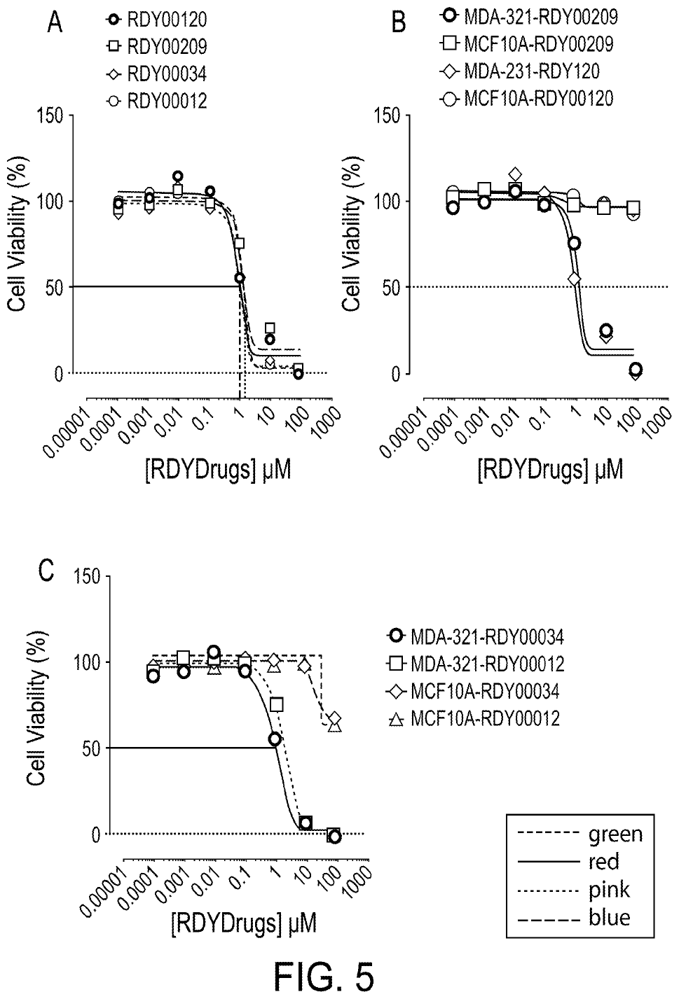

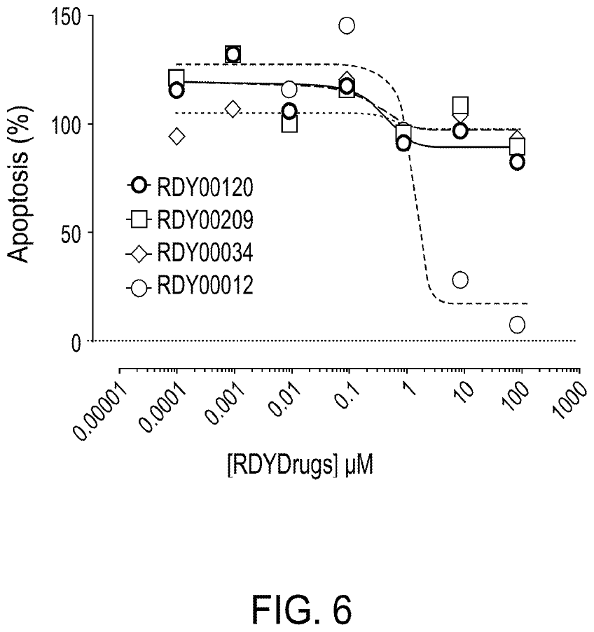

It was found that 4 out of the 7 SMEs-RDY00012 (EC₅₀=7.81 μM), RDY00034 (EC₅₀=5.42 μM), RDY00209 (EC₅₀=6.92 μM) and RDY00120 (EC₅₀=7.14 μM) inhibited TNBC cell viability by 95% (FIG. 5A), in a dose-dependent manner. Interestingly, 2 out of the 4 SMEs, RDY00209 and RDY00120, had no effect on the cell viability of MCF10A cells (FIGS. 5B), suggesting that these 2 SMEs selectively inhibited cell viability only in the cancerous cells. Additionally, RDY00012 & RDY00034 also did not affect the cell viability of MCF10A cells at lower drug concentrations of up to 10 μM (FIG. 5C). These 4 SMEs were further tested for their ability to induce apoptosis in MDA-MB-231 and it was found that neither of them induced apoptosis (FIG. 6), suggesting that these 4 SMEs inhibited cell viability in MDA-MB-231 by necrosis/necroptosis.

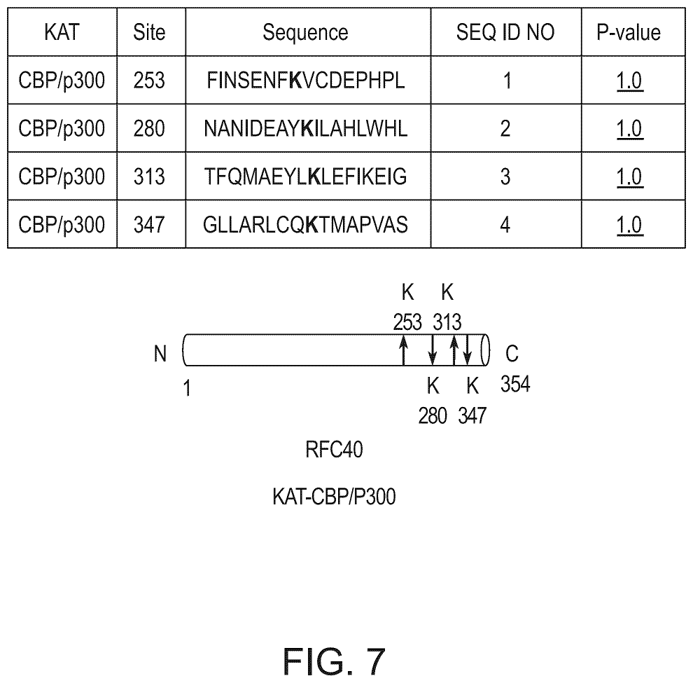

This unique distinction in the inhibition of cell viability in the cancer cells, but not the normal cells, by RDY00209 and RDY00120, was investigated to identify the possible mechanism behind this phenomenon. Without being bound by any theory, it could be possible that RFC40 is undergoing some post-translational modifications (PTM) that are specific to the cancer cells. Proteomic analysis of RFC40 was conducted for post-translational modifications (PTM), such as lysine acetylation, deacetylation, and ubiquitination. The Lysine (K) KAT-specific Acetylation Site Prediction (ASEB) website was used to identify the putative lysine residues that can be acetylated by lysine acetyltransferases (http://bioinfo.bjmu.edu.cn/huac/predict_p/). It was found that RFC40 harbors 4 lysine residues that can be acetylated by CBP/p300 with a perfect score of 1.0, shown in Table 2 below and in FIG. 7.

TABLE 2

| KAT | Site | Sequence | SEQ ID NO | P-value |
|---|---|---|---|---|
| CBP/p300 | 253 | FINSENFKVCDEPHPL | 1 | 1.0 |
| CBP/p300 | 280 | NANIDEAYKILAHLWHL | 2 | 1.0 |
| CBP/p300 | 313 | TFQMAEYLKLEFIKEIG | 3 | 1.0 |
| CBP/p300 | 347 | GLLARLCQKTMAPVAS | 4 | 1.0 |

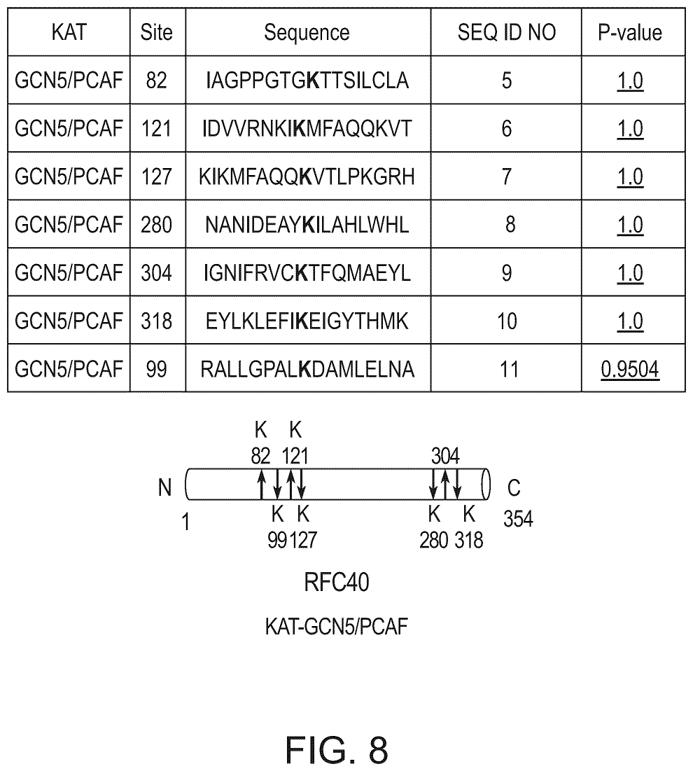

It was also found that RFC40 harbors 6 lysine residues that can be acetylated by GCN5/PCAF lysine acetyltransferases, with a perfect score of 1.0, shown in Table 3 below and FIG. 8.

TABLE 3

| KAT | Site | Sequence | SEQ ID NO | P-value |
|---|---|---|---|---|
| GCN5/PCAF | 82 | IAGPPGTGKTTSILCLA | 5 | 1.0 |
| GCN5/PCAF | 121 | IDVVRNKIKMFAQQKVT | 6 | 1.0 |
| GCN5/PCAF | 127 | KIKMFAQQKVTLPKGRH | 7 | 1.0 |
| GCN5/PCAF | 280 | NANIDEAYKILAHLWHL | 8 | 1.0 |
| GCN5/PCAF | 304 | IGNIFRVCKTFQMAEYL | 9 | 1.0 |
| GCN5/PCAF | 318 | EYLKLEFIKEIGYTHMK | 10 | 1.0 |
| GCN5/PCAF | 99 | RALLGPALKDAMLELNA | 11 | 0.9504 |

Hence it could be possible that RFC40 may be hyper acetylated at the lysine residues by either CBP/p300 or GCN5/PCAF in cancer cells, but not in the normal or non-cancerous cells since both these acetyltransferases have been reported to be overexpressed in breast cancer patients³⁰.

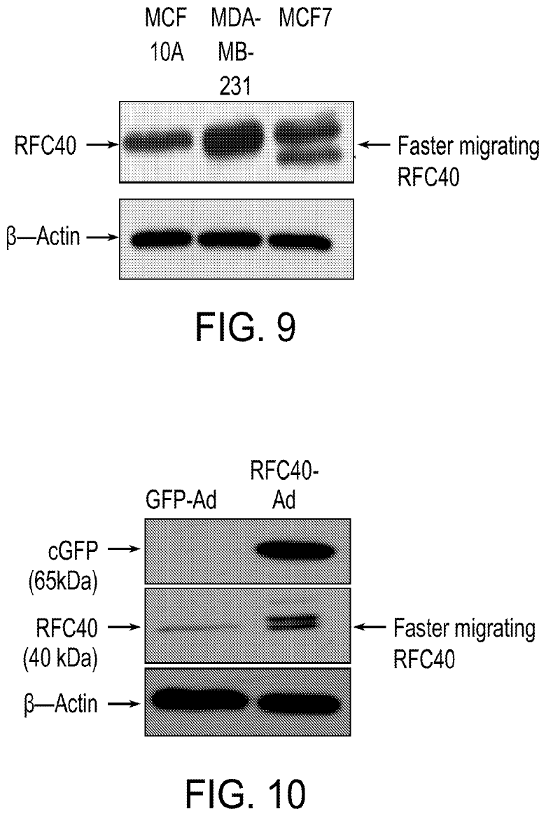

Consistently, an additional faster (lower) migrating band of RFC40 has been observed that appeared only in the breast cancer cells but not in the non-cancerous breast cells (MCF10A)^{23, 26} (FIG. 9). It has been previously demonstrated that over-expression of RFC40 in MCF10A cells induced phenotypic transitions of non-cancerous epithelial breast cells, that are hallmarks of oncogenic transformations. Interestingly, a similar faster (lower) migrating band of RFC40 in MCF10A cells that were transiently transfection with RFC40-Ad full-length gene was observed (FIG. 10),^{23, 26} suggesting that possibly these SMEs are targeting the lysine acetylated RFC40, but not the non-acetylated RFC40. Furthermore, it has been shown that the two processes of lysine acetylation and ubiquitination compete since they share the same lysine residue as substrate. Consequently, lysine acetylation increases the protein stability by preventing its ubiquitination and degradation³¹.

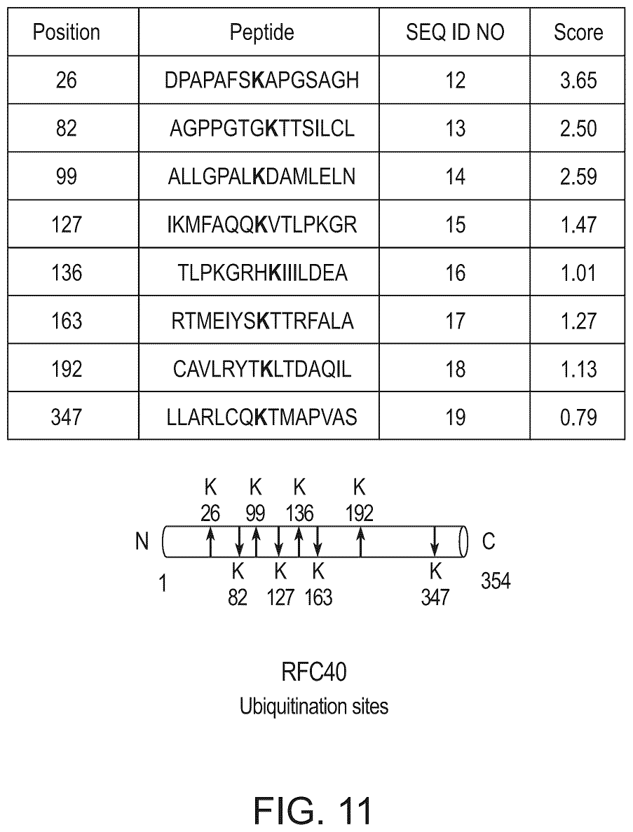

The BDM-PUB site (Prediction of Ubiquitination sites with Bayesian Discrimination Method; http://bdmpub.biocuckoo.org/results.php) was used to identify the putative lysine residues on RFC40 that can be ubiquitinated and 7 lysine residues were found with a high score for ubiquitination, shown in Table 4 below and FIG. 11.

TABLE 4

| Position | Peptide | SEQ ID NO | Score |
|---|---|---|---|
| 26 | DPAPAFSKAPGSAGH | 12 | 3.65 |
| 82 | AGPPGTGKTTSILCL | 13 | 2.50 |
| 99 | ALLGPALKDAMLELN | 14 | 2.59 |
| 127 | IKMFAQQKVTLPKGR | 15 | 1.47 |
| 136 | TLPKGRHKIIILDEA | 16 | 1.01 |
| 163 | RTMEIYSKTTRFALA | 17 | 1.27 |
| 192 | CAVLRYTKLTDAQIL | 18 | 1.13 |
| 347 | LLARLCQKTMAPVAS | 19 | 0.79 |

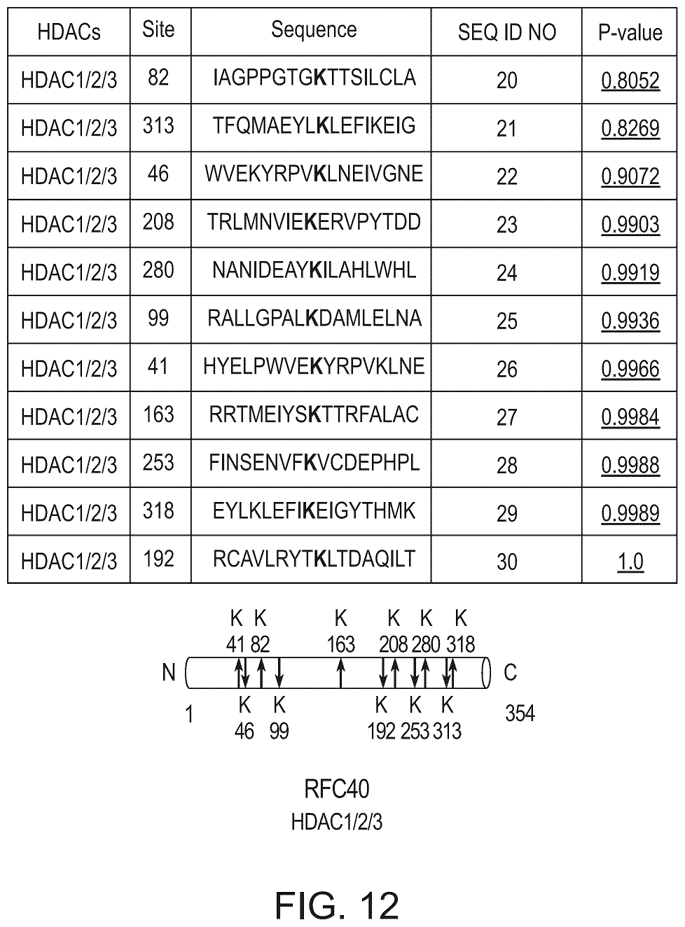

However, a deacetylation event is necessary for the lysine residues to be available for ubiquitination. Therefore, a search was run to identify the lysine residues on RFC40 that can be deacetylated by HDAC1/2/3, using the ASEB website. It was found that 8 lysine residues can be deacetylated by HDAC1/2/3 with the scores ranging from 0.99 to 1.0, shown in Table 5 below and FIG. 12.

TABLE 5

| | | | SEQ | |
|---|---|---|---|---|
| | | | ID | P- |
| HDACs | Site | Sequence | NO | value |
| HDAC1/2/3 | 82 | IAGPPGTGKTTSILCLA | 20 | 0.8052 |
| HDAC1/2/3 | 313 | TFQMAEYLKLEFIKEIG | 21 | 0.8269 |
| HDAC1/2/3 | 46 | WVEKYRPVKLNEIVGNE | 22 | 0.9072 |
| HDAC1/2/3 | 208 | TRLMNVIEKERVPYTDD | 23 | 0.9903 |
| HDAC1/2/3 | 280 | NANIDEAYKILAHLWHL | 24 | 0.9919 |
| HDAC1/2/3 | 99 | RALLGPALKDAMLELNA | 25 | 0.9936 |
| HDAC1/2/3 | 41 | HYELPWVEKYRPVKLNE | 26 | 0.9966 |
| HDAC1/2/3 | 163 | RRTMEIYSKTTRFALAC | 27 | 0.9984 |
| HDAC1/2/3 | 253 | FINSENVFKVCDEPHPL | 28 | 0.9988 |
| HDAC1/2/3 | 318 | EYLKLEFIKEIGYTHMK | 29 | 0.9989 |
| HDAC1/2/3 | 192 | RCAVLRYTKLTDAQILT | 30 | 1.0 |

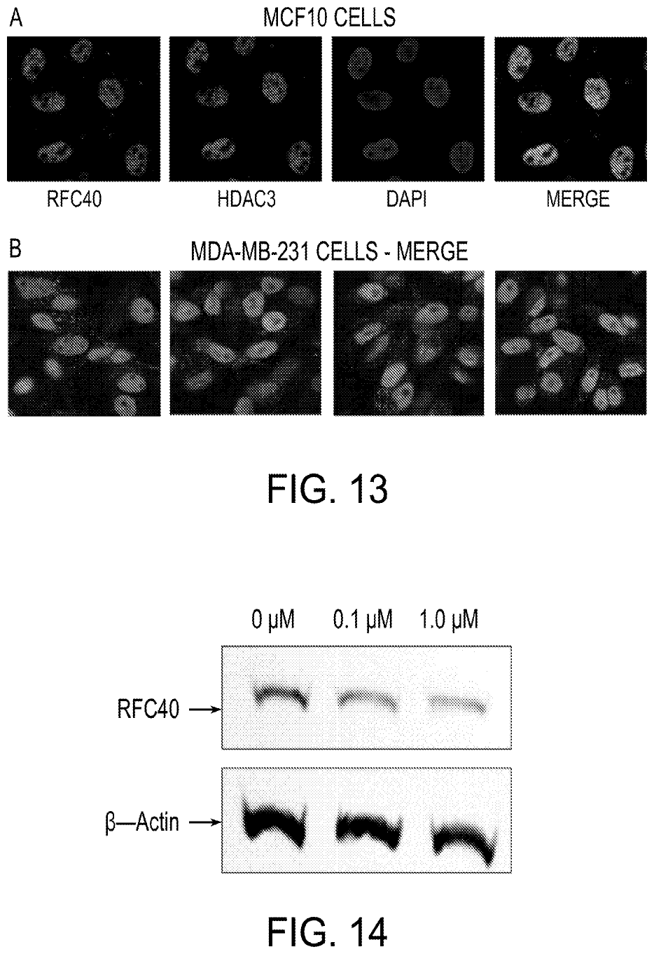

Interestingly, it was found that RFC40 robustly co-localizes with HDAC3, in MCF10A cells (FIG. 13A), however, this co-localization is decreased in the TNBC cells (FIG. 13B), possibly suggesting that deacetylation of RFC40 is decreased in the cancer cells as compared to the non-cancerous cells, thus preventing its ubiquitination in cancer cells, thereby increasing its protein stability. Conversely, there are 2 putative lysine residues, viz., K82 and K99, with high scores for all 3 events of acetylation/deacetylation and ubiquitination, suggesting that these 2 lysine residues can be acetylated/deacetylated and subsequently channelized to the ubiquitin pathway for degradation.

### Example 3

### RFC40 Protein Degradation by RDY00012

### Method

HEK293 cells were plated in 6-well plates and grown until they were 80% confluent. The cells were then treated with RDY00012, in a dose-dependent manner [100 μM, 10 μM, 1 μM, 0.1 μM, 0 μM (DMSO only)], for 24 hours at 37° C. Samples were run 9% SDS-PAGE and Western blot analysis was performed using rabbit polyclonal anti-RFC40 (1:100, Abcepta) and mouse monoclonal anti-β-Actin (1:500, Santa Cruz Tech) primary antibodies, respectively. Following incubation with anti-rabbit, and anti-mouse secondary antibodies, respectively, specific proteins were detected by chemiluminescence.

### Results

HEK293 cells treated with 2 different concentrations of RDY00012 for 24 hr downregulated the expression of RFC40 (FIG. 14), suggesting that the treatment of these cells with RDY00012 potentially accelerated the degradation of RFC40 protein, possibly by its increased ubiquitination.

### Example 4

### Biochemical Assays

Method: Biochemical assays were performed to determine the effects of the 2 SMEs RDY00209 and RDY00120 on the enzymatic activity of RFC40. Recombinant human RFC40 protein was purchased from Origene Technologies (Rockville, MD). ATPase enzyme activity of RFC40 was estimated using the ADP-Glo Max Assay kit³² (Promega, Madison, WI), as per the manufacturer's instructions. RFC40 (250 nM) was pre-incubated, individually, with different concentrations (100 μM, 0.1 μM, 0.1 nM and 0) of RDY00209 and RDY00120 for 30 min and 90 min, respectively, at 37° C. ATP (25 μM) was then added to the mixture and incubated for another 3 hours at 37° C. Luminescence was read on an Envision plate reader.

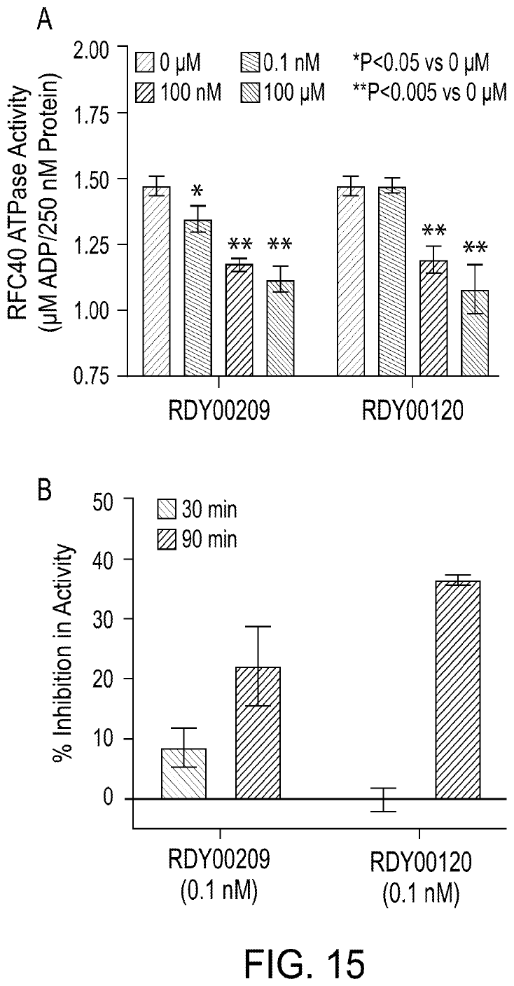

Results: Both RDY00209 and RDY00120 inhibited the enzyme activity in a dose-dependent manner after 30 min of incubation (FIG. 15A). Furthermore, there was a time-dependent (after 90 min) increase in the inhibition of the RFC40 enzyme activity by 22.1% (±6.7%) with 0.1 nM of RDY00209 and 36.5% (±0.7%) with 0.1 nM of RDY00120 (FIG. 15B), suggesting that these 2 SMEs inhibited RFC40 enzyme activity in a time-dependent manner.

### REFERENCES

1. Gupte R S, Weng Y, Liu L, Lee M Y. The second subunit of the replication factor C complex (RFC40) and the regulatory subunit (Rlalpha) of protein kinase A form a protein complex promoting cell survival. Cell Cycle. 2005; 4(2): 323-329.
2. Majka J, Burgers P M. The PCNA-RFC families of DNA clamps and clamp loaders. Prog Nucleic Acid Res Mol Biol. 2004; 78:227-260.
3. Petronczki M, Chwalla B, Siomos M F, Yokobayashi S, Helmhart W, Deutschbauer A M, Davis R W, Watanabe Y, Nasmyth K. Sister-chromatid cohesion mediated by the alternative RF-CCtf18/Dcc1/Ctf8, the helicase Chl1 and the polymerase-alpha-associated protein Ctf4 is essential for chromatid disjunction during meiosis II. J Cell Sci. 2004; 117(Pt 16): 3547-3559.
4. Cai J, Gibbs E, Uhlmann F, Phillips B, Yao N, O'Donnell M, Hurwitz J. A complex consisting of human replication factor C p40, p37, and p36 subunits is a DNA-dependent ATPase and an intermediate in the assembly of the holoenzyme. J Biol Chem. 1997; 272(30):18974-81.
5. Pan Z Q, Chen M, Hurwitz J. The subunits of activator 1 (replication factor C) carry out multiple functions essential for proliferating-cell nuclear antigen-dependent DNA synthesis. Proc Natl Acad Sci U S A. 1993; 90(1):6-10.
6. Ata H, Shrestha D, Oka M, Ochi R, Jong C J, Gebb S, Benjamin J, Schaffer S, Hobart H H, Downey J, McMurtry I, Gupte R. Downregulation of replication factor C-40 (RFC40) causes chromosomal missegregation in neonatal and hypertrophic adult rat cardiac myocytes. PLoS One. 2012;7(6): e39009.
7. Cui J Q, Shi Y F, Zhou H J. Expression of RFC2 and PCNA in different gestational trophoblastic diseases. Chinese J Cancer. 2004; 23:196-200.
8. Staber P B, Linkesch W, Zauner D, Beham-Schmid C, Guelly C, Schauer S, Sill H, Hoefler G. Common alterations in gene expression and increased proliferation in recurrent acute myeloid leukemia. Oncogene. 2004; 23(4):894-904.
9. Merkerova M, Bruchova H, Brdicka R. JNK2 and p38 MAPK over-expressions do not represent key events in chronic myeloid leukemia transformation. Neoplasma. 2007; 54(6):503-10.
10. Xiong S, Wang Q, Zheng L, Gao F, Li J. Identification of candidate molecular markers of nasopharyngeal Carcinoma by tissue microarray and in situ hybridization. Med Oncol. 2011; 28 Suppl 1: S341-8.
11. Nakahara Y, Shiraishi T, Okamoto H, Mineta T, Oishi T, Sasaki K, Tabuchi K. Detrended fluctuation analysis of genome-wide copy number profiles of glioblastomas using array-based comparative genomic hybridization. Neuro Oncol. 2004; 6(4):281-9.
12. Suzuki T, Maruno M, Wada K, Kagawa N, Fujimoto Y, Hashimoto N, Izumoto S, Yoshimine T. Genetic analysis of human glioblastomas using a genomic microarray system. Brain Tumor Pathol. 2004; 21(1):27-34.
13. Ji Z, Li J, Wang J. Up-regulated RFC2 predicts unfavorable progression in hepatocellular carcinoma. Hereditas volume 158, Article number: 17 (2021)
14. McPherson K, Steel C M, Dixon J M. ABC of breast diseases. Breast cancer-epidemiology, risk factors, and genetics. BMJ. 2000; 321(7261):624-8.
15. Misek D E, Kim E H. Protein biomarkers for the early detection of breast cancer. Int J Proteomics. 2011; 2011:343582
16. Normanno N, Morabito A, De Luca A, Piccirillo M C, Gallo M, Maiello M R, Perrone F. Target-based therapies in breast cancer: current status and future perspectives. Endocr Relat Cancer. 2009; 16(3):675-702.
17. Wang D Y, Jiang Z, Ben-David Y, Woodgett J R, Zacksenhaus E. Molecular stratification within triple-negative breast cancer subtypes. Sci Rep. 2019 Dec. 13;9(1):19107.
18. Bauer K R, Brown M, Cress R D, Parise C A, Caggiano V. Descriptive analysis of estrogen receptor (ER)-negative, progesterone receptor (PR)-negative, and HER2-negative invasive breast cancer, the so-called triple-negative phenotype: a population-based study from the California cancer Registry, Cancer. 2007 May 1;109(9):1721-8.
19. Prakash O, Hossain F, Danos D, Lassak A, Scribner R, Miele L. Racial Disparities in Triple Negative Breast Cancer: A Review of the Role of Biologic and Non-biologic Factors. Front Public Health. 2020 Dec. 22;8:576964.
20. Robson M, Im S A, Senkus E, Xu B, Domchek S M, Masuda N, Delaloge S, Li W, Tung N, Armstrong A, Wu W, Goessl C, Runswick S, Conte P. Olaparib for Metastatic Breast Cancer in Patients with a Germline BRCA Mutation. N Engl J Med. 2017 Aug. 10;377(6):523-533. doi: 10.1056/NEJMoa1706450.
21. Wayne Kuznar. PARP Inhibition for TNBC: Single-agent efficacy in patients with triple-negative breast cancer and BRCA mutation. ASCO Reading Room Aug. 29 2017;https://www.medpagetoday.com/reading-room/asco/breast-cancer/67575.
22. Robson M E, Tung N, Conte P, Im S A, Senkus E, Xu B, Masuda N, Delaloge S, Li W, Armstrong A, Wu W, Goessl C, Runswick S, Domchek S M. OlympiAD final overall survival and tolerability results: Olaparib versus chemotherapy treatment of physician's choice in patients with a germline BRCA mutation and HER2-negative metastatic breast cancer. Ann Oncol. 2019 Apr. 1;30(4):558-566. doi: 10.1093/annonc/mdz012.
23. Gupte R. Replication Factor C-40 (RFC40/RFC2) as a prognostic marker and target in Estrogen positive and negative and Triple Negative breast cancer. U.S. Pat. No. 9,546,366. 2017, Jan. 17.
24. Samuel Genheden, Ulf Ryde. The MM/PBSA and MM/GBSA methods to estimate ligand-binding affinities. Expert Opin Drug Discov. 2015 May 4; 10(5): 449-461. doi: 10.1517/17460441.2015.1032936
25. Talele T T, Khedkar S A, Rigby A C. Successful applications of computer aided drug discovery: moving drugs from concept to the clinic. Curr Top Med Chem. 2010;10(1):127-41.
26. Gupte R. Replication Factor C-40 (RFC40/RFC2) as a prognostic marker and target in Estrogen positive and negative and Triple Negative breast cancer. U.S. Pat. No. 9,193,970. 2015, Nov. 24.
27. Overholtzer M, Zhang J, Smolen G A, Muir B, Li W, Sgroi D C, Deng C X, Brugge J S, Haber D A. Transforming properties of YAP, a candidate oncogene on the chromosome 11q22 amplicon. Proc Natl Acad Sci U S A. 2006;103(33):12405-10.
28. Kalluri R, Weinberg R A. The basics of epithelial-mesenchymal transition. J Clin Invest. 2009; 119(6):1420-8.
29. Jaura A I, Flood G, Gallagher H C, Buggy D J. Differential effects of serum from patients administered distinct anaesthetic techniques on apoptosis in breast cancer cells in vitro: a pilot study. Br J Anaesth. 2014 Jul;113 Suppl 1:i63-7.
30. Ramadan W S, Talaat I M, Hachim M Y, Lischka A, Gemoll T, El-Awady R. The impact of CBP expression in estrogen receptor-positive breast cancer. Clin Epigenetics. 2021 Apr. 7;13(1):72.
31. Caron C, Boyault C, Khochbin S. Regulatory crosstalk between lysine acetylation and ubiquitination: role in the control of protein stability. Bioessays. 2005 Apr;27(4):408-15.
32. Veloria J R, Devkota A K, Cho E J, Dalby K N. Optimization of a Luminescence-Based High-Throughput Screening Assay for Detecting Apyrase Activity. SLAS Discov. 2017 Jan;22(1):94-101.

All publications, patents, and patent documents are incorporated by reference herein, as though individually incorporated by reference. This statement of incorporation by reference is intended by applicants, pursuant to 37 C.F.R. § 1.57(b)(1), to relate to each and every individual publication, patent application, or patent, each of which is clearly identified in compliance with 37 C.F.R. § 1.57(b)(2), even if such citation is not immediately adjacent to a dedicated statement of incorporation by reference. The inclusion of dedicated statements of incorporation by reference, if any, within the specification does not in any way weaken this general statement of incorporation by reference. Citation of the references herein is not intended as an admission that the reference is pertinent prior art, nor does it constitute any admission as to the contents or date of these publications or documents. No limitations inconsistent with this disclosure are to be understood therefrom.

The invention has been described with reference to various specific and preferred embodiments and techniques. However, it should be understood that many variations and modifications may be made while remaining within the spirit and scope of the invention.

While specific embodiments have been described above with reference to the disclosed embodiments and examples, such embodiments are only illustrative and do not limit the scope of the invention. Changes and modifications can be made in accordance with ordinary skill in the art without departing from the invention in its broader aspects as defined in the following claims. Such equivalents are intended to be encompassed by the following claims.

### Exemplary Compounds, Methods and Uses are Set Out in the Following Items

Item 1. A compound of Formula I

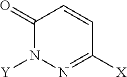

including all pharmaceutically acceptable salts, crystalline forms, stereoisomers, prodrugs, and amorphous forms, wherein
X is selected from the group consisting of aryl and heteroaryl,
   wherein each X is optionally substituted with one or more substituents selected from the group consisting of H, halogen, alkyl, hydroxy, alkoxy, alkenyloxy, alkynyloxy, amido, alkylthiol, alkylsulfoxide, alkylsulfone, alkylthioester, and heteroaryl,
   wherein two adjacent substituents on X may be taken together to form a cycloalkyl, cycloalkenyl, heterocyclyl, and heteroaryl; and
Y is alkyl
   wherein Y is optionally substituted with one or more substituents selected from the group consisting of alkyl, alkenyl, alkynyl, amido, carbonyl, carboxyl, aryl, and heteroaryl,
   wherein two adjacent substituents on Y may be taken together to form a cycloalkyl, cycloalkenyl, heterocyclyl, and heteroaryl.

Item 2. The compound according to item 1, wherein Y is substituted with one or more substituents selected from the group consisting of amido and carboxyl.

Item 3. The compound according to item 2, wherein the amido is substituted with one or more substituents selected from the group consisting of alkyl carboxylic acid, aryl, and heteroaryl.

Item 4. The compound according to item 2, wherein the carboxyl is a carboxylic acid group.

Item 5. A compound of Formula II

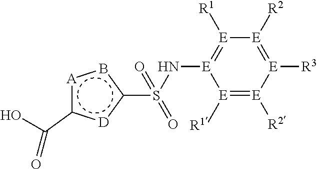

including all pharmaceutically acceptable salts, crystalline forms, stereoisomers, prodrugs, and amorphous forms, wherein
A is selected from the group consisting of CR′, CR′═CH, CR′═N, NH, N, S, and O;
B is selected from the group consisting of CR′, CR′═CH, NH, N, S, and O;
D is selected from the group consisting of CR′, NH, N, S, and O;
E at each instance is independently selected from the group consisting of C and N;
R′ at each instance is independently selected from the group consisting of H, alkyl, alkenyl, alkynyl, hydroxy, alkoxy, and halogen;
R¹ and R¹′ are each individually selected from the group consisting of H, alkyl, alkenyl, alkynyl, hydroxy, alkoxy, alkenyloxy, alkynyloxy, alkylthiol, alkylsulfoxide, alkylsulfone, alkylthioester, halogen, carboxylate, amido, aryl, and heteroaryl;
R² and R²′ are each individually selected from the group consisting of H, alkyl, alkenyl, alkynyl, hydroxy, alkoxy, alkenyloxy, alkynyloxy, alkylthiol, alkylsulfoxide, alkylsulfone, alkylthioester, halogen, carboxylate, amido, aryl, and heteroaryl; and
R³ is selected from the group consisting of H, alkyl, alkenyl, alkynyl, alkoxy, hydroxy, alkenyloxy, alkynyloxy, alkylthiol, alkylsulfoxide, alkylsulfone, alkylthioester, halogen, carboxylate, amido, aryl, and heteroaryl,
   wherein two of R¹, R¹′, R², R²′, and R³ may be taken together to form a cycloalkyl, heterocyclo, aryl, and heteroaryl.

Item 6. The compound according to item 5, wherein
A is CR′, wherein R′ is H;
B is S; and
D is CR′, wherein R′ is H.

Item 7. The compound according to item 5, wherein
A is NH;
B is CR′, wherein R′ is H; and
D is CR′, wherein R′ is H.

Item 8. The compound according to item 5, wherein
A is CR′═CH, wherein R′ is H;
B is CR′, wherein R′ is H; and
D is CR′, wherein R′ is H.

Item 9. The compound according to item 5, wherein
A is CR′, wherein R′ is H;
B is CR′, wherein R′ is H; and
D is O.

Item 10. A compound of Formula III

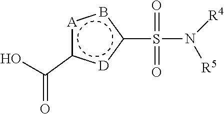

including all pharmaceutically acceptable salts, crystalline forms, stereoisomers, prodrugs, and amorphous forms, wherein
A is selected from the group consisting of CR′, CR′═CH, NH, N, S, and O;
B is selected from the group consisting of CR′, CR′═CH, NH, N, S, and O;
D is selected from the group consisting of CR′, NH, N, S, and O; and
R⁴ and R⁵ may be taken together to form a 5, 6, or 7 membered optionally substituted heterocyclo or a, 6, or 7 membered optionally substituted heteroaryl.

Item 11. A compound of Formula IV

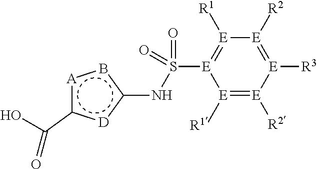

including all pharmaceutically acceptable salts, crystalline forms, stereoisomers, prodrugs, and amorphous forms, wherein
A is selected from the group consisting of CR′, CR′═CH, CR′═N, NH, N, S, and O;
B is selected from the group consisting of CR′, CR′═CH, CR′═N, NH, N, S, and O;
D is selected from the group consisting of CR′, NH, N, S, and O;
E at each instance is independently selected from the group consisting of CR′, NH and N;
R′ at each instance is independently selected from the group consisting of H, alkyl, alkenyl, alkynyl, hydroxy, alkoxy, and halogen;
R¹ and R¹′ are each individually selected from the group consisting of H, alkyl, alkenyl, alkynyl, hydroxy, alkoxy, alkenyloxy, alkynyloxy, alkylthiol, alkylsulfoxide, alkylsulfone, alkylthioester, halogen, carboxylate, amido, aryl, and heteroaryl;
R² and R²′ are each individually selected from the group consisting of H, alkyl, alkenyl, alkynyl, hydroxy, alkoxy, alkenyloxy, alkynyloxy, alkylthiol, alkylsulfoxide, alkylsulfone, alkylthioester, halogen, carboxylate, amido, aryl, and heteroaryl; and
R³ is selected from the group consisting of H, alkyl, alkenyl, alkynyl, alkoxy, hydroxy, alkenyloxy, alkynyloxy, alkylthiol, alkylsulfoxide, alkylsulfone, alkylthioester, halogen, carboxylate, amido, aryl, and heteroaryl,
   wherein two of R¹, R¹′, R², R²′, and R³ may be taken together to form a cycloalkyl, heterocyclo, aryl, and heteroaryl.

Item 12. The compound according to item 11, wherein
A is CR′, wherein R′ is H;
B is S; and
D is CR′, wherein R′ is H.

Item 13. The compound according to item 11, wherein
A is NH;
B is CR′, wherein R′ is H; and
D is CR′, wherein R′ is H.

Item 14. The compound according to item 11, wherein
A is CR′═CH, wherein R′ is H;
B is CR′, wherein R′ is H; and
D is CR′, wherein R′ is H.

Item 15. The compound according to item 11, wherein
A is CR′, wherein R′ is H;
B is CR′, wherein R′ is H; and
D is O.

Item 16. A compound of Formula V

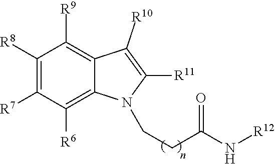

including all pharmaceutically acceptable salts, crystalline forms, stereoisomers, prodrugs, and amorphous forms, wherein
R⁶, R⁷, R⁸, R⁹, R¹⁰, and R¹¹ are each individually selected from the group consisting of H, alkyl, alkenyl, alkynyl, hydroxy, alkoxy, alkenyloxy, alkynyloxy, alkylthiol, alkylsulfoxide, alkylsulfone, alkylthioester, halogen, carboxylate, amido, aryl, and heteroaryl;
R¹² is selected from the group consisting of H, alkyl, alkenyl, alkynyl, alkoxy, hydroxy, alkenyloxy, alkynyloxy, alkylthiol, alkylsulfoxide, alkylsulfone, alkylthioester, halogen, carboxylic acid, carboxylate, amido, aryl, and heteroaryl; and
n is an integer selected from the group consisting of 0, 1, 2, or 3.

Item 17. The compound according to item 16, wherein R¹² is a substituted alkyl group.

Item 18. The compound according to item 17, wherein the substituted alkyl is substituted with an optionally substituted amido group.

Item 19. The compound according to item 18, wherein the optionally substituted alkyl group is substituted with a substituent selected from the group consisting of alkyl carboxylic acid and alkyl-amido carboxylic acid.

Item 20. A compound of Formula VI

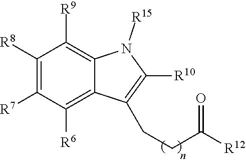

Including all pharmaceutically acceptable salts, crystalline forms, stereoisomers, prodrugs, and amorphous forms, wherein
R⁶, R⁷, R⁸, R⁹, and R¹⁰ are each individually selected from the group consisting of H, alkyl, alkenyl, alkynyl, hydroxy, alkoxy, alkenyloxy, alkynyloxy, alkylthiol, alkylsulfoxide, alkylsulfone, alkylthioester, halogen, carboxylate, amido, aryl, and heteroaryl;
R¹² is selected from the group consisting of H, alkyl, alkenyl, alkynyl, alkoxy, hydroxy, alkenyloxy, alkynyloxy, alkylthiol, alkylsulfoxide, alkylsulfone, alkylthioester, halogen, carboxylic acid, carboxylate, amido, aryl, and heteroaryl;
R¹⁵ is selected from the group consisting of H, alkyl, alkenyl, and alkynyl; and
n is an integer selected from the group consisting of 0, 1, 2, or 3.

Item 21. The compound according to item 20, wherein R¹² is selected from the group consisting of hydroxy and optionally substituted amino.

Item 22. The compound according to item 21, wherein the optionally substituted amino is substituted with an alkylaryl.

Item 23. A compound of Formula VII

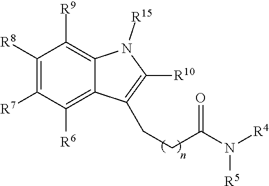

including all pharmaceutically acceptable salts, crystalline forms, stereoisomers, prodrugs, and amorphous forms, wherein
R⁴ and R⁵ are each individually selected from the group consisting of H, alkyl, alkenyl, alkynyl, hydroxy, alkoxy, alkenyloxy, alkynyloxy, alkylthiol, alkylsulfoxide, alkylsulfone, alkylthioester, halogen, carboxylate, amido, aryl, and heteroaryl;
R⁴ and R⁵ may be taken together to form a 5, 6, or 7 membered heterocyclo or a 5, 6, 7 membered heteroaryl;
R⁶, R⁷, R⁹, R⁹, and R¹⁰ are each individually selected from the group consisting of H, alkyl, alkenyl, alkynyl, hydroxy, alkoxy, alkenyloxy, alkynyloxy, alkylthiol, alkylsulfoxide, alkylsulfone, alkylthioester, halogen, carbonyl, carboxylic acid, carboxylate, amido, aryl, and heteroaryl;
R¹⁵ is selected from the group consisting of H, alkyl, alkenyl, and alkynyl; and
n is an integer selected from the group consisting of 0, 1, 2, or 3.

Item 24. The compound according to item 23, wherein the heterocyclo or heteroaryl formed by R⁴ and R⁵ may be substituted with an α, β, γ, or δ—alkylcarboxylic acid.

Item 25. The compound according to item 23, wherein R⁴ and R⁵ taken together yield a piperazine.

Item 26. A compound of Formula VIII

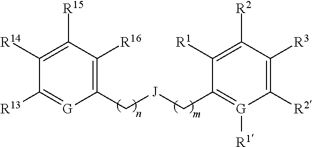

including all pharmaceutically acceptable salts, crystalline forms, stereoisomers, prodrugs, and amorphous forms, wherein
G each instance is individually selected from the group consisting of CH and N;
J is selected from the group consisting of CH, NR′, O, and S;
   R′ is selected from the group consisting of H and alkyl;
   R¹, R¹′, R², R²′, and R³ are each individually selected from the group consisting of H, alkyl, alkenyl, alkynyl, hydroxy, alkoxy, alkenyloxy, alkynyloxy, alkylthiol, alkylsulfoxide, alkylsulfone, alkylthioester, halogen, carbonyl, carboxylic acid, carboxylate, cyano, amido, aryl, and heteroaryl;
   R¹³, R¹⁴, R¹⁵, and R¹⁶ are each individually selected from the group consisting of H, alkyl, alkenyl, alkynyl, carboxylic acid, and carboxylate; and
   m and n are each individually selected from an integer selected from the group consisting of 0, 1, 2, or 3.

Item 27. The compound according to item 26, m and n are each individually an integer selected from the group consisting of 0 or 1.

Item 28. The compound according to item 26, wherein
m is 0; and
n is 1.

Item 29. The compound according to item 26, wherein
m is 1; and
n is 0.

Item 30. A pharmaceutical composition comprising a compound of according to any one of items 1-29 or an enantiomer, diastereomer, hydrate, solvate, or a pharmaceutically acceptable salt thereof, and at least one pharmaceutically acceptable carrier or excipient.

Item 31. A method of inhibiting the expression and/or activity of RCF40 in a subject, comprising administering to the subject a compound according to any one of items 1-29 or an enantiomer, diastereomer, hydrate, solvate, or a pharmaceutically acceptable salt thereof, wherein the activity of RCF40 activity is inhibited in the subject.

Item 32. A method of ameliorating or treating RCF40-mediated cancers in a subject, comprising administering to the subject an effective amount of a compound according to any one of items 1-29 or an enantiomer, diastereomer, hydrate, solvate, or a pharmaceutically acceptable salt thereof, wherein the growth of cancer cells is inhibited in a subject.

Item 33. The method of item 31, wherein the cancers are breast cancer, kidney cancer, choriocarcinoma, acute myeloid leukemia, chronic myeloid leukemia, nasopharyngeal cancer, glioblastomas, prostate cancer, cervical cancer, ovarian cancer, uterine cancer, and hepatocellular carcinoma.

Item 34. The method of item 32, wherein the breast cancer is estrogen sensitive breast cancer, HER2 positive breast cancer or triple negative breast cancer.

Item 35. A compound for use in a method of inhibiting the expression and/or activity of RCF40 in a subject, wherein the method comprises administering to the subject a compound according to any one of items 1-29 or an enantiomer, diastereomer, hydrate, solvate, or a pharmaceutically acceptable salt thereof, wherein the activity of RCF40 activity is inhibited in the subject.

Item 36. A compound for use in a method of ameliorating or treating RCF40-mediated cancers in a subject, wherein the method comprises administering to the subject a compound according to any one of items 1-29 or an enantiomer, diastereomer, hydrate, solvate, or a pharmaceutically acceptable salt thereof, wherein the growth of cancer cells is inhibited in a subject.

1-34. (canceled)

35. A pharmaceutical composition comprising a compound of Formula II 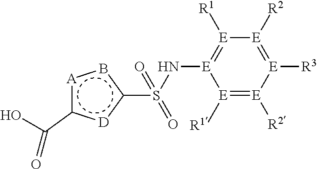
including all pharmaceutically acceptable salts, crystalline forms, stereoisomers, prodrugs, and amorphous forms, wherein
A is selected from the group consisting of CR′, CR′═CH, CR′═N, NH, N, S, and O;
B is selected from the group consisting of CR′, CR′═CH, NH, N, S, and O;
D is selected from the group consisting of CR′, NH, N, S, and O;
E at each instance is independently selected from the group consisting of C and N;
R′at each instance is independently selected from the group consisting of H, alkyl, alkenyl, alkynyl, hydroxy, alkoxy, and halogen;
R¹ and R¹′ are each individually selected from the group consisting of H, alkyl, alkenyl, alkynyl, hydroxy, alkoxy, alkenyloxy, alkynyloxy, alkylthiol, alkylsulfoxide, alkylsulfone, alkylthioester, halogen, carboxylate, amido, aryl, and heteroaryl;
R² and R²′ are each individually selected from the group consisting of H, alkyl, alkenyl, alkynyl, hydroxy, alkoxy, alkenyloxy, alkynyloxy, alkylthiol, alkylsulfoxide, alkylsulfone, alkylthioester, halogen, carboxylate, amido, aryl, and heteroaryl; and
R³ is selected from the group consisting of H, alkyl, alkenyl, alkynyl, alkoxy, hydroxy, alkenyloxy, alkynyloxy, alkylthiol, alkylsulfoxide, alkylsulfone, alkylthioester, halogen, carboxylate, amido, aryl, and heteroaryl,
wherein two of R¹, R¹′, R², R²′, and R³ may be taken together to form a cycloalkyl, heterocyclo, aryl, and heteroaryl, and
at least one pharmaceutically acceptable carrier or excipient.

36. The pharmaceutical composition according to claim 35, wherein
A is CR′, wherein R′is H;
B is S; and
D is CR′, wherein R′is H.

37. The pharmaceutical composition according to claim 35, wherein
A is NH;
B is CR′, wherein R′is H; and
D is CR′, wherein R′is H.

38. The pharmaceutical composition according to claim 35, wherein
A is CR′═CH, wherein R′is H;
B is CR′, wherein R′is H; and
D is CR′, wherein R′is H.

39. The pharmaceutical composition according to claim 35, wherein
A is CR′, wherein R′is H;
B is CR′, wherein R′is H; and
D is O.

40. The pharmaceutical composition according to claim 35, wherein the compound is represented by the structure: 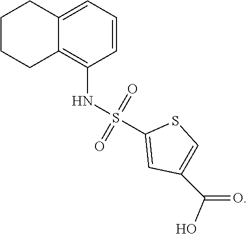 [SMILES: O=C(O)c1csc(S(=O)(=O)Nc2cccc3c2CCCC3)c1]
or a pharmaceutically acceptable salt, crystalline form, amorphous form or prodrug thereof.

41. The pharmaceutical composition according to claim 35, wherein the composition comprises:
a water-miscible vehicle selected from the group consisting of ethyl alcohol, polyethylene glycol, and polypropylene glycol;
a non-aqueous vehicle selected from the group consisting of corn oil, cottonseed oil, peanut oil, sesame oil, ethyl oleate, isopropyl myristate, and benzyl benzoate; or
a combination thereof.

42. A pharmaceutical composition comprising a compound of Formula V 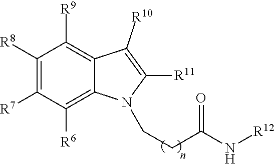
including all pharmaceutically acceptable salts, crystalline forms, stereoisomers, prodrugs, and amorphous forms, wherein
R⁶, R⁷, R⁸, R⁹, R¹⁰, and R¹¹ are each individually selected from the group consisting of H, alkyl, alkenyl, alkynyl, hydroxy, alkoxy, alkenyloxy, alkynyloxy, alkylthiol, alkylsulfoxide, alkylsulfone, alkylthioester, halogen, carboxylate, amido, aryl, and heteroaryl;
R¹² is selected from the group consisting of H, alkyl, alkenyl, alkynyl, alkoxy, hydroxy, alkenyloxy, alkynyloxy, alkylthiol, alkylsulfoxide, alkylsulfone, alkylthioester, halogen, carboxylic acid, carboxylate, amido, aryl, and heteroaryl; and
n is an integer selected from the group consisting of 0, 1, 2, or 3,
and at least one pharmaceutically acceptable carrier or excipient.

43. The pharmaceutical composition according to claim 42, wherein R¹² is a substituted alkyl group.

44. The pharmaceutical composition according to claim 43, wherein the substituted alkyl is substituted with an optionally substituted amido group.

45. The pharmaceutical composition according to claim 44, wherein the optionally substituted alkyl group is substituted with a substituent selected from the group consisting of alkyl carboxylic acid and alkyl-amido carboxylic acid.

46. The pharmaceutical composition according to claim 42, wherein the compound is represented by the structure: 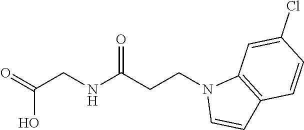 [SMILES: O=C(O)CNC(=O)CCn1ccc2ccc(Cl)cc21]
or a pharmaceutically acceptable salt, crystalline form, amorphous form or prodrug thereof.

47. The pharmaceutical composition according to claim 42, wherein the composition comprises:
a water-miscible vehicle selected from the group consisting of ethyl alcohol, polyethylene glycol, and polypropylene glycol;
a non-aqueous vehicle selected from the group consisting of corn oil, cottonseed oil, peanut oil, sesame oil, ethyl oleate, isopropyl myristate, and benzyl benzoate; or
a combination thereof.

48. A method of inhibiting the expression and/or activity of RCF40 in a subject, comprising administering to the subject a pharmaceutical composition according to claim 35, wherein the expression and/or activity of RCF40 activity is inhibited in the subject.

49. A method of ameliorating or treating RCF40-mediated cancers in a subject, comprising administering to the subject an effective amount of a pharmaceutical composition according to claim 35, wherein the growth of cancer cells is inhibited in a subject.

50. The method of claim 49, wherein the cancers are breast cancer, glioblastomas, cervical cancer, ovarian cancer, uterine cancer, acute myeloid leukemia, chronic myeloid leukemia, kidney cancer, nasopharyngeal cancer, prostate cancer, hepatocellular carcinoma, or choriocarcinoma.

51. The method of claim 50, wherein the cancer is breast cancer, wherein the breast cancer is estrogen/progesterone sensitive breast cancer, HER2 positive breast cancer or triple negative breast cancer.

52. The method of claim 49, wherein the administering is intra-tumoral, oral or parenteral.

53. A method of inhibiting the expression and/or activity of RCF40 in a subject, comprising administering to the subject a pharmaceutical composition according to claim 42, wherein the expression and/or activity of RCF40 activity is inhibited in the subject.

54. A method of ameliorating or treating RCF40-mediated cancers in a subject, comprising administering to the subject an effective amount of a pharmaceutical composition according to claim 42, wherein the growth of cancer cells is inhibited in a subject.

55. The method of claim 54, wherein the cancers are breast cancer, glioblastomas, cervical cancer, ovarian cancer, uterine cancer, acute myeloid leukemia, chronic myeloid leukemia, kidney cancer, nasopharyngeal cancer, prostate cancer, hepatocellular carcinoma, or choriocarcinoma.

56. The method of claim 55, wherein the cancer is breast cancer, wherein the breast cancer is estrogen/progesterone sensitive breast cancer, HER2 positive breast cancer or triple negative breast cancer.

57. The method of claim 54, wherein the administering is intra-tumoral, oral or parenteral.
